(19) United States
(12) Patent Application Publication
(10) Pub. No.: US 2018/0179483 A1
(43) Pub. Date: Jun. 28, 2018

(54) IMAGING APPARATUS FOR EXPERIMENTAL USE, EXPERIMENTAL SYSTEM, AND CONTROL METHOD OF EXPERIMENTAL SYSTEM

(71) Applicant: Olympus Corporation, Tokyo (JP)

(72) Inventors: Hiroki Amino, Hino-shi (JP); Takato Aoki, Hino-shi (JP); Osamu Nonaka, Sagamihara-shi (JP)

(21) Appl. No.: 15/842,776

(22) Filed: Dec. 14, 2017

(30) Foreign Application Priority Data
Dec. 27, 2016 (JP) 2016-252545

(51) Int. Cl. C12M 1/00; H04N 5/232

(52) U.S. Cl. CPC C12M 23/50; H04N 5/23296; C12M 29/26; C12M 43/00; H04N 5/23203

(57) ABSTRACT

An experimental system includes an imaging unit which generates image data by imaging a sample, and an observation moving mechanism which changes a position of the imaging unit. The system also includes a supply and removal unit which supplies a liquid to the sample or removes the liquid from the sample, an operation moving mechanism which changes a position of the supply and removal unit, and a pump which moves the liquid through the supply and removal unit. Furthermore, the system includes a control circuit which controls operations of the observation moving mechanism and the operation moving mechanism such that the positions of the imaging unit and the supply and removal unit change together, and which controls an operation of the pump in accordance with the positions of the imaging unit and the supply and removal unit.

### CROSS-REFERENCE TO RELATED APPLICATIONS

This application is based upon and claims the benefit of priority from prior Japanese Patent Application No. 2016-252545, filed Dec. 27, 2016, the entire contents of which are incorporated herein by reference.

### BACKGROUND OF THE INVENTION

### 1. Field of the Invention

The present invention relates to an imaging apparatus for experimental use, an experimental system, and a control method of the experimental system.

### 2. Description of the Related Art

In general, known in the art is an apparatus wherein a culture vessel is statically placed in an incubator and an image of cultured cells or the like in the culture vessel is taken. For example, Jpn. Pat. Appin. KOKAI Publication No. 2005-295818 discloses a technique related to an apparatus which takes a number of images while moving a camera (imaging unit) inside an incubator so as to take images of cells existing in a wide range of a culture vessel.

In general, an experiment may be conducted in which cells are cultured in a culture vessel, a reagent is added to a culture medium inside the culture vessel, and how the cultured cells change is observed. To culture the cells, regular replacement of the culture medium is required.

### BRIEF SUMMARY OF THE INVENTION

According to one embodiment of the present invention, an imaging apparatus for experimental use, which is movable to different positions in a specific plane in which a sample is distributed, and which enables an imaging position to be changed in accordance with a position of an external substance operation mechanism configured to add or remove a substance, said imaging apparatus comprising an imaging unit which generates image data by imaging the sample, an observation moving mechanism which changes a position of the imaging unit, and a control circuit which controls an operation of the observation moving mechanism such that the position of the imaging unit and a substance operation position of the substance operation mechanism change together.

According to one embodiment of the present invention, an experimental system comprising an imaging unit which generates image data by imaging a sample, an observation moving mechanism which changes a position of the imaging unit, a supply and removal unit which supplies a liquid to the sample or removes the liquid from the sample, an operation moving mechanism which changes a position of the supply and removal unit; a pump which moves the liquid through the supply and removal unit, and a control circuit which controls an operation of the observation moving mechanism and an operation of the operation moving mechanism such that the position of the imaging unit and the position of the supply and removal unit change together, and which controls an operation of the pump in accordance with the position of the imaging unit and the position of the supply and removal unit.

According to one embodiment of the present invention, a method for controlling an experimental system which includes an imaging unit which generates image data by imaging a sample, an observation moving mechanism which changes a position of the imaging unit, a supply and removal unit which supplies a liquid to the sample or removes a liquid from the sample, an operation moving mechanism which changes a position of the supply and removal unit, and a pump which moves the liquid through the supply and removal unit, said method comprising controlling an operation of the observation moving mechanism and an operation of the operation moving mechanism such that the position of the imaging unit and the position of the supply and removal unit change together, and controlling an operation of the pump in accordance with the position of the imaging unit and the position of the supply and removal unit.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic diagram illustrating an experimental system according to an embodiment of the present invention.

FIG. 2A is a block diagram schematically illustrating an exemplary configuration of an observation apparatus according to an embodiment.

FIG. 2B is a block diagram schematically illustrating an exemplary configuration of a liquid operation apparatus according to an embodiment.

FIG. 2C is a block diagram schematically illustrating an exemplary configuration of an operation apparatus according to an embodiment.

FIG. 2D is a block diagram schematically illustrating an exemplary configuration of a display apparatus according to an embodiment.

FIG. 3 is a block diagram schematically illustrating an exemplary configuration of an experimental system according to an embodiment.

FIG. 4 is a flowchart illustrating an overview of how an operation apparatus is controlled according to an embodiment.

FIG. 5 is a diagram schematically illustrating an example of an operation screen displayed on an operation apparatus according to an embodiment.

FIG. 6 is a flowchart illustrating an overview of an example of how specific position observation mode processing is performed according to an embodiment.

FIG. 7 is a flowchart illustrating an overview of how measurement mode processing is performed according to an embodiment.

FIG. 8A is a flowchart illustrating an overview of an example of how liquid operation mode processing is performed according to an embodiment.

FIG. 8B is a flowchart illustrating an overview of an example of how liquid operation mode processing is performed according to an embodiment.

FIG. 9 is a diagram schematically illustrating an example of an operation screen displayed on an operation apparatus according to an embodiment.

FIG. 10 is a flowchart illustrating an overview of an example of how automatic culture medium replacement mode processing is performed according to an embodiment.

FIG. 11A is a flowchart illustrating an overview of an example of how an observation apparatus is controlled according to an embodiment.

FIG. 11B is a flowchart illustrating an overview of an example of how an observation apparatus is controlled according to an embodiment.

FIG. 12A is a flowchart illustrating an overview of an example of how a liquid operation apparatus is controlled according to an embodiment.

FIG. 12B is a flowchart illustrating an overview of an example of how a liquid operation apparatus is controlled according to an embodiment.

FIG. 13 is a flowchart illustrating an overview of an example of how a display apparatus is controlled according to an embodiment.

### DETAILED DESCRIPTION OF THE INVENTION

One embodiment of the present invention will be described. For example, an experimental system 1 according to the present embodiment can acquire images of cells while culturing the cells. In addition, the experimental system 1 can add a reagent to the culture medium or replace the culture medium while culturing the cells. The experimental system 1 enables an observation apparatus for acquiring images of the cells to be remotely controlled by wireless communications and enables acquired images to be displayed on a distant display apparatus by wireless communications. Furthermore, the experimental system 1 enables a liquid operation apparatus for adding a reagent to the culture medium to be remotely controlled by wireless communications. Therefore, the experimental system 1 enables observation of cells and replacement of the culture medium, with the cells statically kept in the incubator. The experimental system 1 may be used inside the incubator or in other places such as inside a clean bench.

[Configuration of Experimental System]

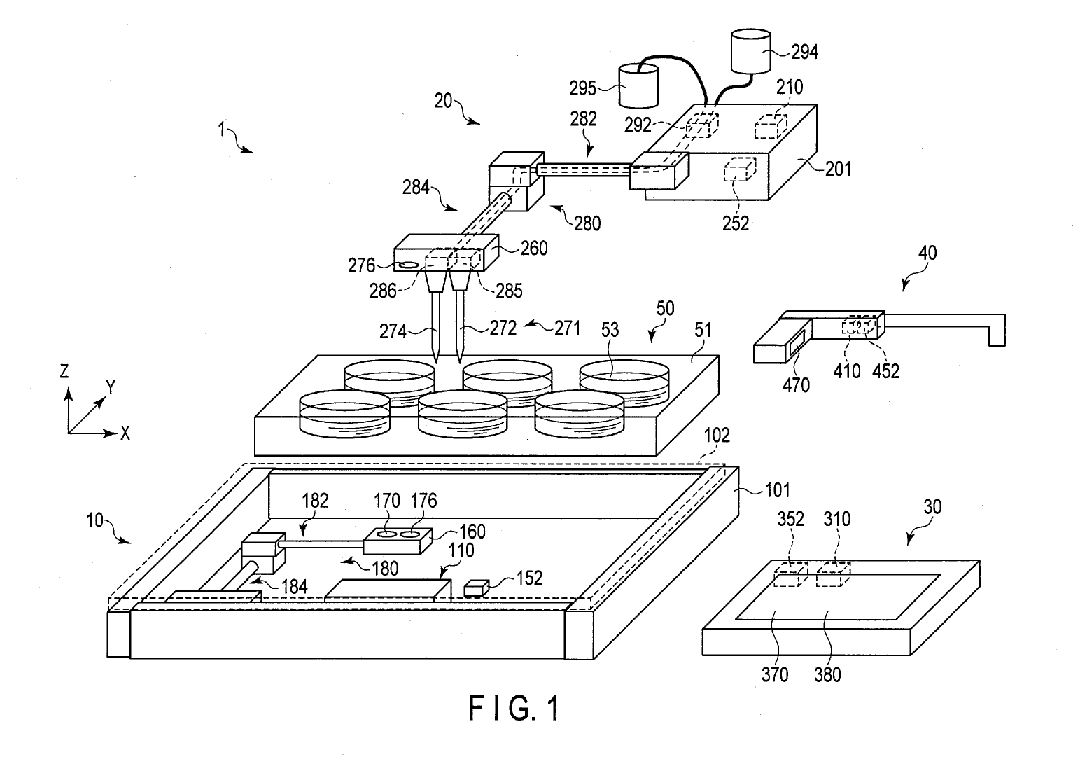

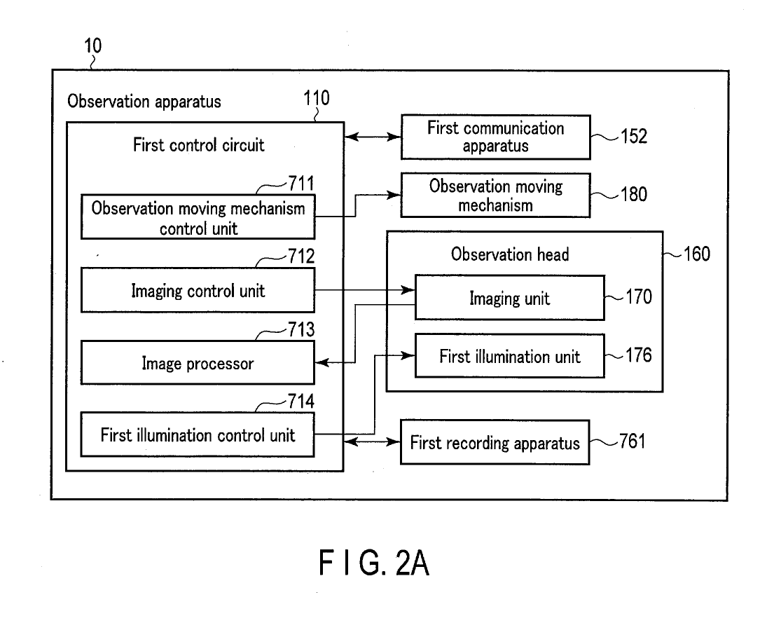

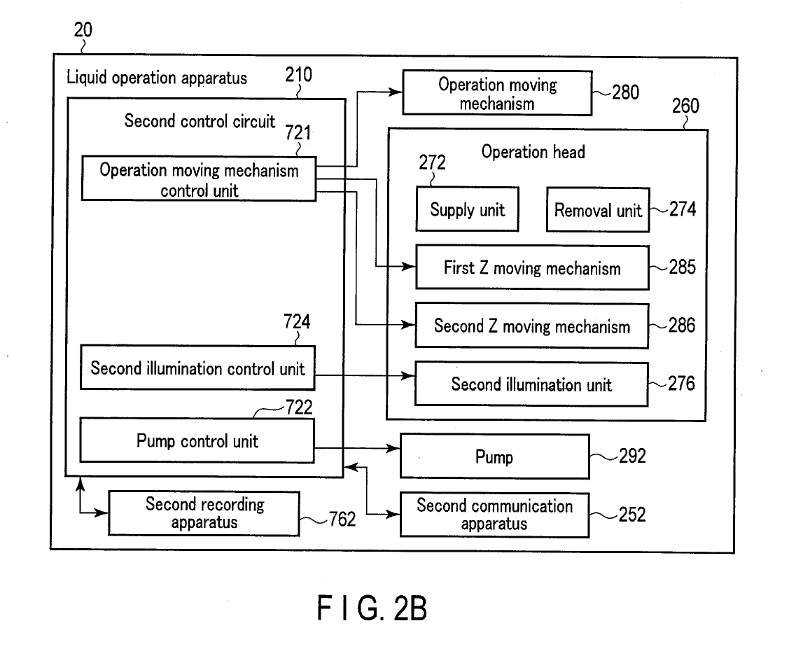

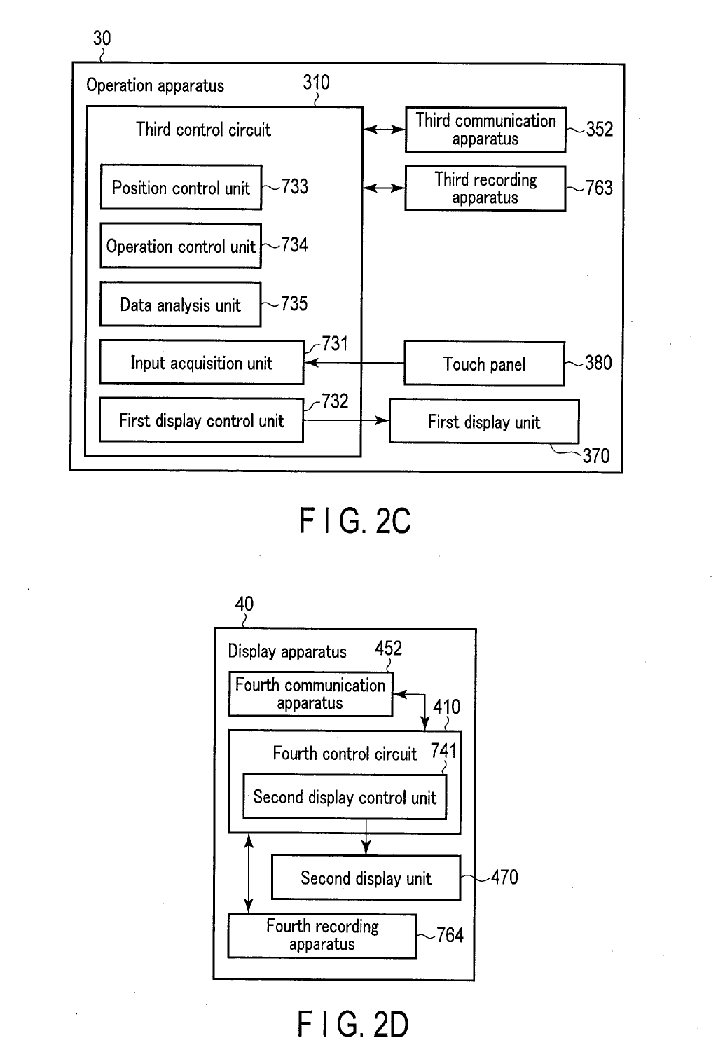

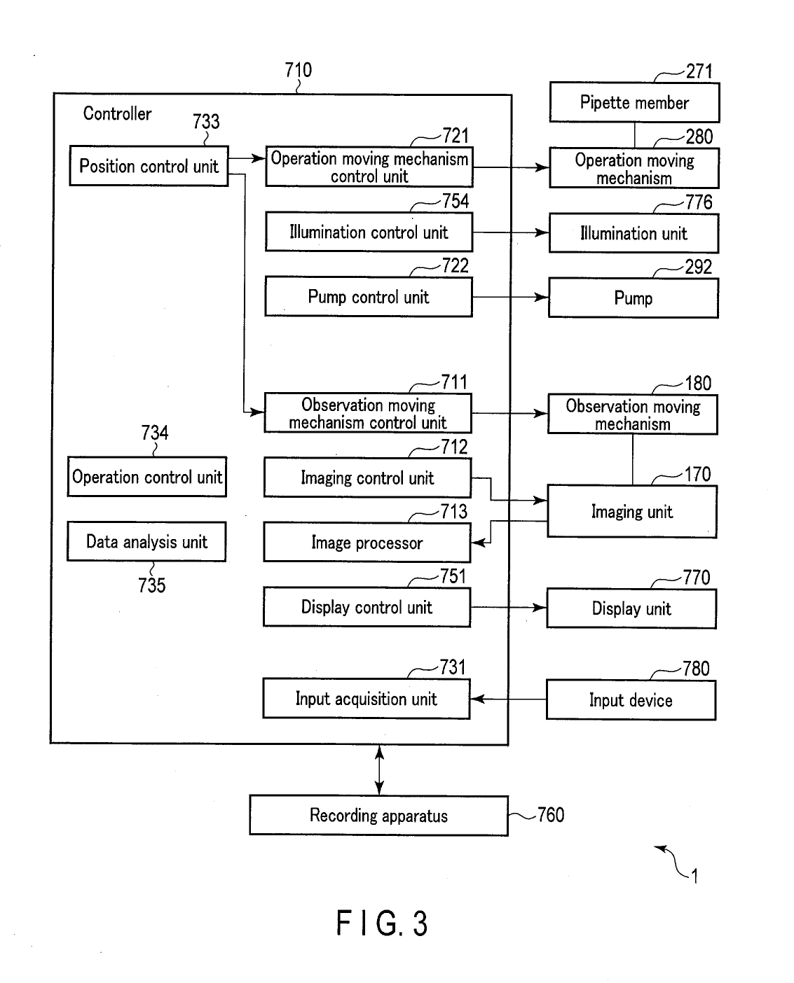

FIG. 1 is a schematic diagram showing an overview of how the experimental system 1 looks like. As shown in FIG. 1, the experimental system 1 is provided with an observation apparatus 10, a liquid operation apparatus 20, an operation apparatus 30 and a display apparatus 40. FIG. 2A is a block diagram showing an example of a configuration of the observation apparatus 10, FIG. 2B is a block diagram showing an example of a configuration of the liquid operation apparatus 20, FIG. 2C is a block diagram showing an example of a configuration of the operation apparatus 30, and FIG. 2D is a block diagram showing an example of a configuration of the display apparatus 40. FIG. 3 is a block diagram showing an overview of the function of the entire experimental system 1.

<Observation Apparatus>

As shown in FIG. 1, the profile of the observation apparatus 10 has substantially a flat plate shape. The observation apparatus 10 is provided, for example, inside an incubator, and a sample 50 to be observed is arranged on the horizontal top plate of the observation apparatus 10. For the sake of explanation, an X-axis and a Y-axis perpendicular to each other are defined in a plane parallel to the surface of the observation apparatus 10 on which the sample 50 is arranged, and a Z-axis is defined as an upward vertical axis perpendicular to both the X-axis and the Y-axis. A transparent plate 102 is placed on top of the observation apparatus 10, and an imaging unit 170 is provided inside the casing 101 of the observation apparatus 10. A sample 50 is statically placed on this transparent plate 102. The observation apparatus 10 takes an image of the sample 50, with the transparent plate 102 interposed, and the image of the sample 50 is acquired thereby. Although the word “observation” may be generally associated with visual inspection, the observation apparatus 10 can perform machine observation and obtain observation results as numerical values, data, or the like. The observation apparatus 10 may be regarded as one type of imaging apparatus.

An example of the sample 50 to be observed by the experimental system 1 will be described. A culture medium 53 is put into the vessel 51, and cells or the like are cultured in the culture medium 53. The vessel 51 is, for example, a petri dish, a culture flask, or a multiwell plate. The vessel 51 is, for example, a culture vessel for culturing a living specimen. The vessel 51 is not limited to any specific shape or size. The lid of the vessel 51 reflects light. The cells to be measured may be cells of any kind, including adhesive cells and floating cells. The cells may be spheroids or tissues. In addition, the cells may be derived from any living thing or may be germs or the like. As described above, the sample 50 includes a living sample which is either the living thing itself or is derived from the living thing. The experimental system 1 is not limitedly used for a living sample. The experimental system can be used in experiments for a variety of substances. For example, the experimental system 1 can be used for observing the manner in which various substances are added or removed. For example, the experimental system 1 can be used for making researches into the manner in which a specific reagent diffuses when it is added.

Various components of the observation apparatus 10 are provided inside the casing 101 of the observation apparatus 10. The interior of an incubator is in a hot and humid environment; it has a temperature of 37° C. and a humidity of 95%. Since the observation apparatus 10 is used in such a hot and humid environment, the interior of the observation apparatus 10 is hermetically sealed by the casing 101 and the transparent plate 102.

An imaging unit 170 is provided on an observation head 160 of the casing 101. The imaging unit 170 includes an image sensor, an imaging optical system, etc. The imaging unit 170 takes an image of the region where the sample 50 is present and thus acquires an image of the sample 50. A first illumination unit 176 is provided on the observation head 160. The first illumination unit 176 includes a light source, such as an LED, an illumination optical system, etc., and radiates illumination light toward the sample 50. The illumination light radiating from the first illumination unit 176 is reflected by the lid of the sample 50, irradiates observation targets, such as cells, and falls on the optical system of the imaging unit 170. In this manner, the imaging unit 170 can acquire an image of the sample 50 in the illuminated state.

The observation head 160 is moved by an observation moving mechanism 180. The observation moving mechanism 180 is provided with an X moving mechanism 182 including, for example, a feed screw and an actuator for moving the observation head 160 in the X-axis direction. The moving mechanism 180 is also provided with a Y moving mechanism 184 including, for example, a feed screw and an actuator for moving the observation head 160 in the Y-axis direction. The imaging unit 170 can take an image of the sample 50 on the transparent plate 102 in an enlarged scale, but the image that can be taken at a time is limited to part of the sample 50. However, by moving the imaging unit 170 with the observation moving mechanism 180, the imaging unit 170 can acquire an image of a wide range. The imaging position in the Z-axis direction can be changed by changing the in-focus position of the imaging optical system of the imaging unit 170 in the direction of the optical axis. In place of the focus adjustment mechanism or in combination therewith, the observation moving mechanism 180 maybe provided with a Z moving mechanism including a feed screw and an actuator for moving the observation head 160 in the Z-axis direction.

A first control circuit 110 for controlling the observation moving mechanism 180 and imaging unit 170 is provided inside the casing 101. A first communication apparatus 152 is also provided inside the casing 101. The first communication apparatus 152 is an apparatus which communicates with the liquid operation apparatus 20, operation apparatus 30 and display apparatus 40. The communications are wireless communications using, for example, Wi-Fi, Bluetooth, or the like. Where Wi-Fi is used, for example, the first communication apparatus 152 of the observation apparatus 10 functions as an access point. Part or all of the wireless communications may be replaced with wired communications.

As shown in FIG. 2A, the observation apparatus 10 (imaging apparatus) is provided with a first recording apparatus 761, in addition to the above-mentioned first control circuit 110, the imaging unit 170 and first illumination unit 176 provided on the observation head 160, the observation moving mechanism 180 and the first communication apparatus 152. The first control circuit 110 functions as an observation moving mechanism control unit 711, an imaging control unit 712, an image processor 713 and a first illumination control unit 714. The observation moving mechanism control unit 711 controls the operation of the observation moving mechanism 180. The imaging control unit 712 controls the operation performed by the imaging unit 170. The image processor 713 performs various kinds of image processing for the image acquired by the imaging unit 170. In addition to the above, the first control circuit 110 has the function of controlling each portion of the observation apparatus 10 and the function of controlling the communications performed by the first communication apparatus 152. The first recording apparatus 761 stores various kinds of information, including the programs and parameters used by the first control circuit 110. The first recording apparatus 761 stores various kinds of information prepared by the first control circuit 110. The first illumination control unit 714 controls the operation of the first illumination unit 176.

<Liquid Operation Apparatus>

As shown in FIG. 1, the liquid operation apparatus 20 has the function of adding a reagent to a culture medium of the sample 50 statically placed on the observation apparatus 10 and the function of replacing the liquid culture medium 53 of the sample 50.

The liquid operation apparatus 20 is provided with an operation head 260 and an operation moving mechanism 280. The operation head 260 is moved by an operation of the operation moving mechanism 280. For example, the operation head 260 can be moved to a position where it is opposed to the observation head 160 of the observation apparatus 10. The operation head 260 is provided with a supply and removal unit 271 and a second illumination unit 276. The supply and removal unit 271 includes a supply unit 272 and a removal unit 274. The supply and removal unit 271 includes a pipette-like member, for example. The pipette-like member mentioned here is not limited to a pipette but can include a funnel, a dropper, a syringe needle, or the like. That is, the pipette-like member covers all members that can add or remove a substance and may simply be a tube, a groove, a hole or the like. The supply and removal unit 271 can function as a substance addition unit, a substance removal unit, a substance addition/removal unit, a substance changing unit, a substance moving unit, a substance position changing unit, or a device including these units in combination. The supply and removal unit 271 includes members and mechanisms for realizing a substance changing unit that adds a predetermined substance to a sample or removes it from the sample, a substance replacement unit that removes a predetermined substance from a sample and then adds another substance, a substance operation unit that moves a predetermined substance in the sample, or a device including these units in combination.

The supply unit 272 is configured to add a liquid to the vessel 51 of the sample 50 and is shaped, for example, like a pipette. The supply unit 272 moves upward or downward in the Z-axis direction, and the tip end of the supply unit 272 enters the culture medium 53 or is taken away from the culture medium 53. To move the supply unit 272 in the Z-axis direction, the operation head 260 is provided with a first Z moving mechanism 285.

The removal unit 274 is configured to remove a liquid from the vessel 51 of the sample 50 and is shaped, for example, like a pipette. The removal unit 274 moves upward or downward in the Z-axis direction, and the tip end of the removal unit 274 enters the culture medium 53 or is taken away from the culture medium 53. To move the removal unit 274 in the Z-axis direction, the operation head 260 is provided with a second Z moving mechanism 286.

The second illumination unit 276 radiates illumination light toward the sample 50 to illuminate the sample 50. The illumination light radiating from the second illumination unit 276 passes through the sample 50 and is incident on the imaging unit 170 provided on the observation head 160 opposed to the operation head 260. In this manner, the imaging unit 170 can acquire an image of the sample 50 in the illuminated state. When the liquid in the vessel 51 of the sample 50 is treated by means of the supply unit 272 or removal unit 274, the lid of the sample 50 is kept removed. In this case, the observation target (e.g., cells) of the sample 50 cannot be illuminated with the light radiating from the first illumination unit 176 provided for the observation head 160, so that the second illumination unit 276 is used.

The position of the illumination light can be used for the positional adjustment between the observation head 160 and the operation head 260. To be specific, the position of the illumination light radiating from the second illumination unit 276 can be specified based on the image taken by the imaging unit 170. By utilization of the specified position of the illumination light, the observation head 160 and the operation head 260 may be adjusted in position such that they are opposed to each other. In connection with the present embodiment, the observation head 160 and the operation head 260 are adjusted in position by the operation apparatus 30 detailed later, but this is not restrictive. For example, the observation apparatus 10 may analyze the position of the illumination light radiating from the second illumination unit 276, based on the image the observation apparatus 10 acquires, and may move the observation head 160 to the position of the illumination light. In this case, the observation apparatus 10 operates in such a manner as to follow the operation of the liquid operation apparatus 20. Alternatively, the liquid operation apparatus 20 may acquire, from the observation apparatus 10, the image acquired by the observation apparatus 10. In this case, the liquid operation apparatus 20 may analyze the positional relationship between the observation head 160 and the operation head 260 based on where the position of the illumination light radiating from the second illumination unit 276 is in the image, and may move the position of the operation head 260. In this case, the liquid operation apparatus 20 operates in such a manner as to follow the operation of the observation apparatus 10. Where the observation apparatus 10 operates in accordance with the operation of the liquid operation apparatus 20, the following is available. For example, the liquid operation apparatus 20 may identify an instruction based on the color, wavelength, etc. of the light radiating from the second illumination unit 276. Further, the observation apparatus 10 may identify information representing that the observation head 160 is located near the operation head 260 and other instructions, using a predetermined ON/OFF pattern of the light radiating from the second illumination unit 276. The observation head 160 may be controlled to be at the position where the light from the second illumination unit 276 is bright, e.g., at the position where the observation head 160 is opposed to the operation head 260, or to be at another position. The second illumination unit 276 may emit an illumination beam having an asymmetric shape pattern. In this case, which portion of the detected illumination beam is identified, and the observation head 160 is moved in the X direction or Y direction in accordance with the shape of the illumination beam. For example, where the left-side pattern is detected by the imaging unit, the observation head 160 is moved to a position where it can detect a more rightward portion of the beam, thereby enabling position adjustment. In addition, the positions can be shifted on purpose. Prior to the position control of the observation head 160, the observation apparatus 10 may request that the liquid operation apparatus 20 emit illumination light as required. Similarly, the liquid operation apparatus 20 may control the position of the operation head 260 in accordance with the position of the observation head 160.

The operation moving mechanism 280 is provided with an X moving mechanism 282 including, for example, a feed screw and an actuator for moving the operation head 260 in the X-axis direction. The operation moving mechanism 280 is also provided with a Y moving mechanism 284 including, for example, a feed screw and an actuator for moving the operation head 260 in the Y-axis direction. Since the operation head 260 can be moved by the operation moving mechanism 280, the operation head 260 can add or remove a reagent at various positions of the sample 50 placed on the transparent plate 102 of the observation apparatus 10.

The liquid operation apparatus 20 is provided with a pump 292. The liquid operation apparatus 20 is also provided with a reagent container 294 connected to the supply unit 272 through a tube and a waste liquid container 295 connected to the removal unit 274 through a tube. The pump 292 feeds the reagent in the reagent container 294 to the supply unit 272. The pump 292 drains the liquid from the removal unit 274 to the waste liquid container 295. Although only one reagent container 294 and only one waste liquid container 295 are depicted in FIG. 1, a plurality of these containers may be employed, and the liquid operation apparatus 20 may be configured to selectively supply liquids.

A second control circuit 210, which is used for controlling the operation moving mechanism 280, first Z moving mechanism 285, second Z moving mechanism 286, second illumination unit 276 and pump 292, is provided in the casing 201 of the liquid operation apparatus 20. A second communication apparatus 252, used for performing communications with the first communication apparatus 152 of the observation apparatus 10, etc., is also provided in the casing 201. The communications are wireless communications using, for example, Wi-Fi, Bluetooth, or the like.

As shown in FIG. 2B, the liquid operation apparatus 20 is provided with a second recording apparatus 762, in addition to the above-mentioned second control circuit 210, operation head 260, operation moving mechanism 280, pump 292 and second communication apparatus 252. The second control circuit 210 functions as an operation moving mechanism control unit 721, a pump control unit 722 and a second illumination control unit 724. The operation moving mechanism control unit 721 controls the operation of the operation moving mechanism 280. The operation moving mechanism control unit 721 also controls the operations of the first Z moving mechanism 285 and second Z moving mechanism 286 of the operation head 260. The pump control unit 722 controls the operation of the pump 292. The second illumination control unit 724 controls the operation of the second illumination unit 276. In addition to the above, the second control circuit 210 has the function of controlling each portion of the liquid operation apparatus 20 and the function of controlling the communications performed by the second communication apparatus 252. The second recording apparatus 762 stores various kinds of information, including programs and parameters used by the second control circuit 210, and also stores various kinds of information prepared by the second control circuit 210.

In connection with the present embodiment, the liquid operation apparatus 20 will be described as having the function of adding a reagent to a culture medium of the sample 50 and the function of replacing the liquid culture medium 53 of the sample 50, and a description will be given of how a liquid is operated. It should be noted, however, that the liquid operation apparatus 20 can be replaced with a substance operation apparatus including a substance operation mechanism capable of operating various kinds of materials and substances, including solids and gases. For example, the substance operation apparatus may perform the replacement, addition or removal of a gas or perform the addition, removal or movement of a solid included in a liquid or a gas. As described above, the sample 50 handled by the experimental system 1 is not limited to a living sample but may be various kinds of samples that can be the subjects of various experiments. Therefore, the substance operation apparatus can operate various kinds of materials or substances which can be the subjects of various experiments.

<Operation Apparatus>

As shown in FIG. 1, the operation apparatus 30 is, for example, a tablet type information terminal, a smartphone, a personal computer (PC) or a dedicated information terminal. In FIG. 1, a tablet type information terminal is depicted. The operation apparatus 30 is placed on the outside of the incubator. The operation apparatus 30 performs communications with the observation apparatus 10. The operation apparatus 30 may perform communications with the liquid operation apparatus 20 or the display apparatus 40. The operation apparatus 30 controls the operations of the observation apparatus 10, liquid operation apparatus 20, display apparatus 40, etc.

The operation apparatus 30 is provided with a first display unit 370, such as a liquid crystal display, and a touch panel 380. The operation apparatus may include an input device, such as a switch, a dial, a keyboard or a mouse, other than the touch panel 380.

A third communication apparatus 352 is provided for the operation apparatus 30. The third communication apparatus 352 is an apparatus which communicates with the first communication apparatus 152, etc. For example, the observation apparatus 10 and the operation apparatus 30 communicate with each other through the first communication apparatus 152 and the third communication apparatus 352.

As shown in FIG. 2C, the operation apparatus 30 is provided with a third recording apparatus 763, in addition to the third control circuit 310, third communication apparatus 352, first display unit 370 and touch panel 380. The third control circuit 310 controls each portion of the operation apparatus 30. The third recording apparatus 763 stores, for example, programs and various parameters used by the third control circuit 310. The third recording apparatus 763 also stores data obtained by the observation apparatus 10 and received from the observation apparatus 10, data created based on the data received from the observation apparatus 10, etc.

The third control circuit 310 functions as an input acquisition unit 731, a first display control unit 732, a position control unit 733, an operation control unit 734, a data analysis unit 735, etc. The input acquisition unit 731 acquires data relating to an input to the touch panel 380 from the touch panel 380, for example. The first display control unit 732 controls the operation of the first display unit 370. To integratedly control the position of the observation head 160 of the observation apparatus 10 and the position of the operation head 260 of the liquid operation apparatus 20, the position control unit 733 generates control signals to be supplied to the observation moving mechanism control unit 711 and operation moving mechanism control unit 721 and outputs the generated control signals. To integratedly control the operations of the imaging unit 170 of the observation apparatus 10 and the second illumination unit 276, pump 292, etc. of the liquid operation apparatus 20, the operation control unit 734 generates control signals to be supplied to the observation apparatus 10 and liquid operation apparatus 20 and outputs the generated control signals. The data analysis unit 735 performs various kinds of data analysis, for example, by using the data obtained by the observation apparatus 10 and acquired from the third communication apparatus 352.

<Display Apparatus>

The experimental system 1 may comprise a display apparatus different from the first display unit 370 of the operation apparatus 30. As shown in FIG. 1, the experimental system 1 in the present embodiment is provided with a wearable display apparatus 40, such as a head-mounted display. The display apparatus 40 is provided with a second display unit 470 arranged in front of the user's eyes. Preferably, the second display unit 470 is configured to permit the user to visually recognize both the normal field of view and an image displayed on the second display unit 470 by using, for example, a pupil division method. For example, an image taken by the observation apparatus 10 is displayed on the second display unit 470. As a result, the user is allowed to understand the situation of the sample 50 when conducting work.

The display apparatus 40 is provided with a fourth control circuit 410 and a fourth communication apparatus 452. As shown in FIG. 2D, the fourth control circuit 410 has the function of a second display control unit 741. The second display control unit 741 controls the display operation of the second display unit 470. The fourth communication apparatus 452 is configured to communicate with the first communication apparatus 152, etc. The display apparatus 40 is provided with a fourth recording apparatus 764. The fourth recording apparatus 764 stores, for example, programs and various parameters used by the fourth control circuit 410.

<Others>

Each of the first control circuit 110, second control circuit 210, third control circuit 310 and fourth control circuit 410 incorporates an integrated circuit including a central processing unit (CPU), an application specific integrated circuit (ASIC), a field programmable gate array (FPGA), or the like. Each of these control circuits may be constituted by a single integrated circuit or by a combination of a number of integrated circuits. Each of the first recording apparatus 761, second recording apparatus 762, third recording apparatus 763 and fourth recording apparatus 764 includes such a recording apparatus as a semiconductor memory or a hard disk.

The configurations shown in FIGS. 2A to 2D are just examples, and the functions described above maybe attained by other elements of the observation apparatus 10, liquid operation apparatus 20, operation apparatus 30 and display apparatus 40. For example, the data analysis does not have to be performed by the operation apparatus 30; it may be performed by the observation apparatus 10. The operations of each portions of the liquid operation apparatus 20 may be controlled by the first control circuit 110 of the observation apparatus 10. The display apparatus 40 may be omitted. In this case, all images maybe displayed on the first display unit 370 of the operation apparatus 30.

As an example of the communications between the observation apparatus 10, liquid operation apparatus 20, operation apparatus 30 and display apparatus 40, reference was made to the case where the first communication apparatus 152 of the observation apparatus 10 is an access point in the infrastructure mode of Wi-Fi communications and communicates with the other communication apparatuses. However, the communications are not limited to this example. A communication apparatus other than the first communication apparatus 152 may be an access point, and the ad-hoc mode may be used. The observation apparatus 10, liquid operation apparatus 20, operation apparatus 30 and display apparatus 40 maybe connected to such a network as the Internet. In this case, communications are performed between the apparatuses through the network. Communications using Bluetooth may be performed.

As described above, the above-mentioned functions can be assigned to the observation apparatus 10, liquid operation apparatus 20, operation apparatus 30 and display apparatus 40 in a variety of manners. What is only required is that the experimental system 1 as a whole has the above-mentioned functions. FIG. 3 is a function block diagram of the whole experimental system 1. The experimental system 1 is provided with an imaging unit 170, an observation moving mechanism 180, a supply and removal unit 271, an operation moving mechanism 280 and a pump 292. The experimental system 1 is also provided with an input device 780 corresponding to the touch panel 380, a display unit 770 corresponding to both the first display unit 370 and the second display unit 470, and an illumination unit 776 corresponding to both the first illumination unit 176 and the second illumination unit 276.

The experimental system 1 is provided with a recording apparatus 760 corresponding to the first recording apparatus 761, second recording apparatus 762, third recording apparatus 763 and fourth recording apparatus 764. The experimental system 1 is provided with the function of a controller 710. The controller 710 functions as an observation moving mechanism control unit 711, an imaging control unit 712, an image processor 713, an operation moving mechanism control unit 721, a pump control unit 722, a position control unit 733, an operation control unit 734 and a data analysis unit 735. The controller 710 has the function of a display control unit 751 corresponding to both the first display control unit 732 and the second display control unit 741. The controller 710 has the function of an illumination control unit 754 corresponding to both the first illumination control unit 714 and the second illumination control unit 724. The controller 710 has the function of a display control unit 751 corresponding to both the first display control unit 732 and the second display control unit 741.

[Operation of Experimental System]

How the experimental system 1 operates will be described. In the following, a description will be given of the case where the experimental system 1 has four operation modes although the operations of the experimental system 1 are not limited to them. The operation modes of the experimental system 1 are a specific position observation mode, a measurement mode, a liquid operation mode and an automatic culture medium replacement mode.

The specific position observation mode is a mode in which the observation head 160 of the observation apparatus 10 is moved in accordance with the user's operation and an image of the region intended by the user is acquired. Using the specific position observation mode, the user can observe a desired position of the sample 50 at a desired point of time.

The measurement mode is a mode in which the observation head 160 of the observation apparatus 10 moves in accordance with a predetermined pattern and a necessary image is acquired thereby. In various experiments, images of the sample 50 have to be taken according to predetermined procedures and analysis has to be made based on the images obtained according to the procedures. In the measurement mode, the user can acquire data in a predetermined method by performing a simple operation, with no need to attend to details. The measurement mode may be started in response to a user's instruction, as described in connection with the present embodiment, or may be started when a preset time comes.

The liquid operation mode is a mode in which a liquid is supplied to the sample 50 or removed from the sample 50 in response to the user's operation. To be more specific, in the liquid operation mode, the observation head 160 of the observation apparatus 10 and the operation head 260 of the liquid operation apparatus 20 move together based on a user's instruction, and the liquid operation apparatus 20 supplies or removes a liquid. In the liquid operation mode, the user can add a reagent to a desired position of the sample 50 and perform replacement of a culture medium.

The automatic culture medium replacement mode is a mode in which all culture mediums of the sample 50 are replaced. In the automatic culture medium replacement mode, the observation head 160 of the observation apparatus 10 and the operation head 260 of the liquid operation apparatus 20 move together in accordance with a predetermined pattern and culture mediums are removed and supplied. In the meantime, the experimental system 1 makes various kinds of determination, based on the images obtained by the imaging unit 170. In an experiment in which cells are cultured, the culture mediums have to be regularly replaced. By using the experimental system 1, the user can replace the culture mediums without manual operations.

<Operation of Operation Apparatus>

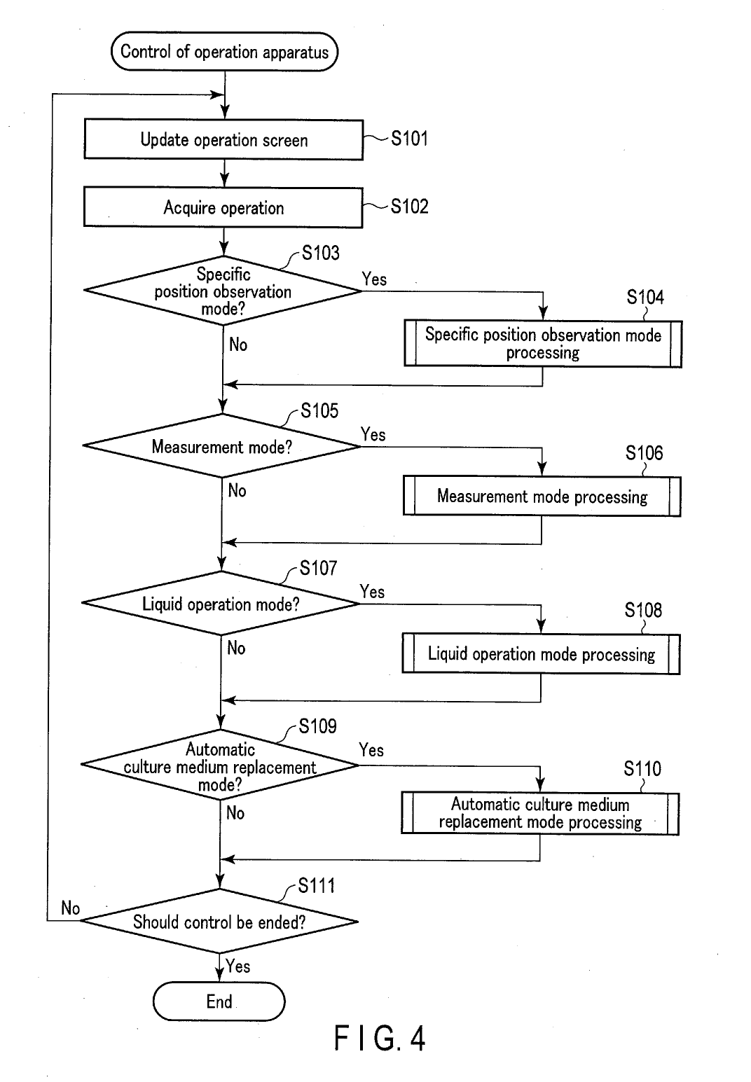

FIG. 4 is a flowchart illustrating an overview of an example of how the operation apparatus 30 operates. The operation apparatus 30 outputs an instruction for controlling the operation of each portion of the experimental system 1.

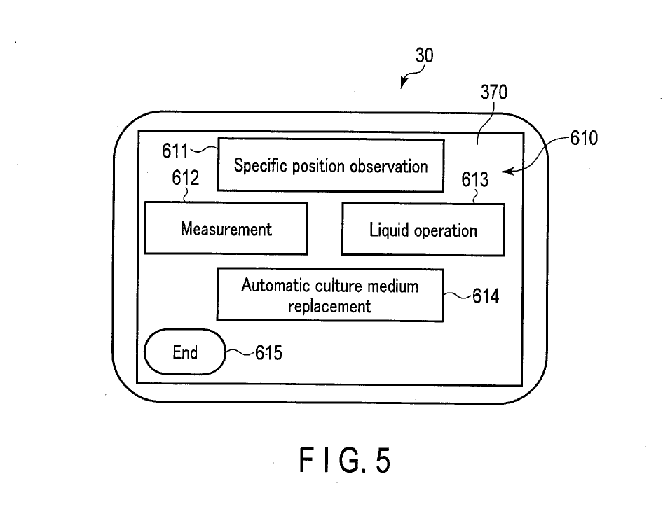

In step S101, the third control circuit 310 updates the operation screen displayed on the first display unit 370. For example, such an image as shown in FIG. 5 is displayed on the first display unit 370 of the operation apparatus 30. That is, the operation screen 610 including, for example, a specific position observation icon 611, a measurement icon 612, a liquid operation icon 613, an automatic culture medium replacement icon 614 and an end icon 615 is displayed on the first display unit 370. The specific position observation icon 611 is an icon for selecting the specific position observation mode. The measurement icon 612 is an icon for selecting the measurement mode. The liquid operation icon 613 is an icon for selecting the liquid operation mode. The automatic culture medium replacement icon 614 is an icon for selecting the automatic culture medium replacement mode. The end icon 615 is an icon for selecting the end of the control of the experimental system 1.

In step S102, the third control circuit 310 acquires an operation input by the user from the touch panel 380. For example, the touch panel 380 detects which icon, the specific position observation icon 611, the measurement icon 612, the liquid operation icon 613, the automatic culture medium replacement icon 614, or the end icon 615, is touched. Based on the obtained operation, determinations described below are made and various kinds of processing are performed.

In step S103, the third control circuit 310 determines whether or not the specific position observation mode is selected. Unless the specific position observation mode is selected, the process proceeds to step S105. If the specific position observation mode is selected, the process proceeds to step S104. In step S104, the third control circuit 310 performs specific position observation mode processing. After the specific position observation mode processing, the process proceeds to step S105.

In step S105, the third control circuit 310 determines whether or not the measurement mode is selected. Unless the measurement mode is selected, the process proceeds to step S107. If the measurement mode is selected, the process proceeds to step S106. In step S106, the third control circuit 310 performs the measurement mode processing. After the measurement mode processing, the process proceeds to step S107.

In step S107, the third control circuit 310 determines whether or not the liquid operation mode is selected. Unless the liquid operation mode is selected, the process proceeds to step S109. If the liquid operation mode is selected, the process proceeds to step S108. In step S108, the third control circuit 310 performs the liquid operation mode processing. After the liquid operation mode processing, the process proceeds to step S109.

In step S109, the third control circuit 310 determines whether or not the automatic culture medium replacement mode is selected. Unless the automatic culture medium replacement mode is selected, the process proceeds to step S111. If the automatic culture medium replacement mode is selected, the process proceeds to step S110. In step S110, the third control circuit 310 performs automatic culture medium replacement mode processing. After the automatic culture medium replacement mode processing, the process proceeds to step S111.

In step S111, the third control circuit 310 determines whether or not the operation apparatus control should be ended. If an input instructing the termination of the operation apparatus control processing is entered, for example, when the user touches the end icon 615, it is determined that the operation apparatus control processing should be terminated. The operation apparatus control is then terminated. If it is determined that the operation apparatus control should not be terminated, the process returns to step S101.

A description will be given of how the operation apparatus 30 operates in each mode.

(Specific Position Observation Mode Processing)

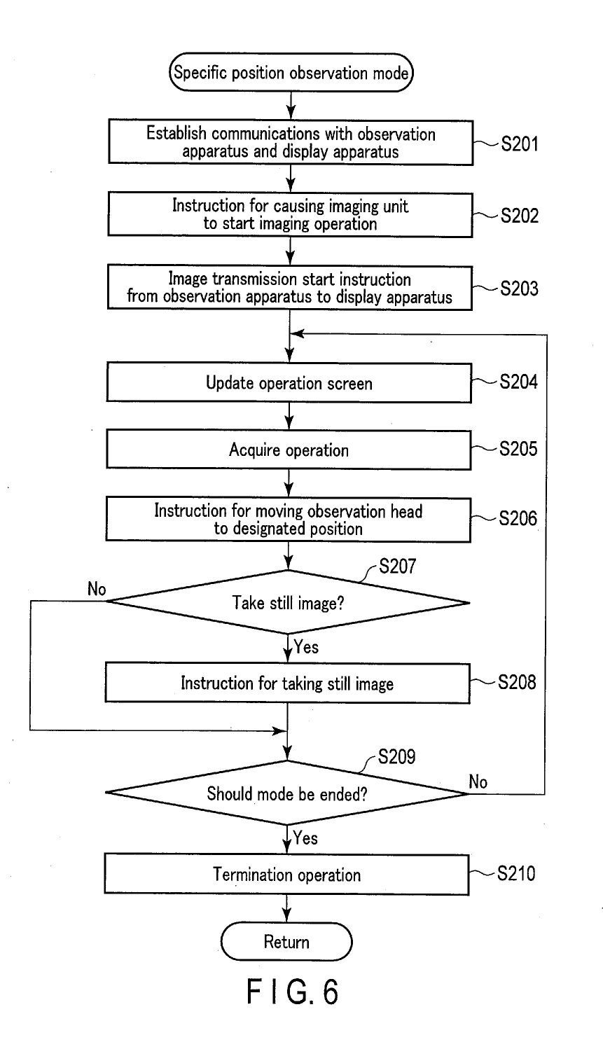

An example of the specific position observation mode processing performed in step S104 will be described, referring to the flowchart shown in FIG. 6. The specific position observation mode processing is processing in which the observation head 160 of the observation apparatus 10 is moved in accordance with the user's operation and an image of the region intended by the user is acquired by the imaging unit 170.

In step S201, the third control circuit 310 establishes communications among the operation apparatus 30, the observation apparatus 10 and the display apparatus 40. For example, Wi-Fi communications are established between the observation apparatus 10 and the operation apparatus 30 and between the observation apparatus 10 and the display apparatus 40. If necessary, Bluetooth communications may be established between the operation apparatus 30 and the display apparatus 40.

In step S202, the third control circuit 310 supplies the observation apparatus 10 with an instruction for causing the imaging unit 170 to start an imaging operation. As a result, the imaging unit 170 of the observation apparatus 10 starts the imaging operation.

In step S203, the third control circuit 310 supplies the observation apparatus 10 with an instruction for causing the observation apparatus 10 to send the image obtained by the imaging operation to the display apparatus 40. As a result, the observation apparatus 10 starts sending the image obtained by the imaging unit 170 to the display apparatus 40. Upon receipt of the image, the display apparatus 40 displays the received image on the second display unit 470. By this display operation, the user can confirm the image currently acquired by the observation apparatus 10, as a live view image.

In step S204, the third control circuit 310 updates the operation screen displayed on the first display unit 370. The updated operation screen includes: a direction indication icon indicating the direction in which an observation target is present, i.e., the direction in which the imaging unit 170 should be moved; a focus indication icon for designating the coordinates of the region to be imaged and for moving the in-focus plane of the imaging unit 170; an imaging instruction icon for taking a still image; etc.

In step S205, the third control circuit 310 acquires an operation input by the user from the touch panel 380. In step S206, the third control circuit 310 supplies the observation apparatus 10 with an instruction for moving the observation head 160 of the observation apparatus 10 to the designated position corresponding to the acquired operation. As a result, the observation head 160 moves to the designated position.

In step S207, the third control circuit 310 determines whether or not an instruction for taking a still image is entered by the user. Unless the instruction for taking a still image is entered, the process proceeds to step S209. If the instruction for taking a still image is entered, the process proceeds to step S208.

In step S208, the third control circuit 310 supplies the observation apparatus 10 with an instruction that causes the imaging unit 170 to perform a still image photographing operation. As a result, the imaging unit 170 of the observation apparatus 10 performs a still image photographing operation, and a still image is acquired thereby. The acquired still image is transmitted to the display apparatus 40. The second display unit 470 of the display apparatus 40 displays the received image. Because of the operations mentioned above, the user can confirm the still image acquired by the observation apparatus 10. The acquired still image may be stored in the first recording apparatus 761 of the observation apparatus 10; alternatively, it may be transmitted to the operation apparatus 30 and stored in the third recording apparatus 763 of the operation apparatus 30. After the still image acquisition processing, the process proceeds to step S209.

In step S209, the third control circuit 310 determines whether or not the specific position observation mode should be ended. For example, if the user enters an instruction to end the specific position observation mode processing, then it is determined that the specific position observation mode processing should be ended. At the time, the process proceeds to step S210. If it is determined that the specific position observation mode processing should not be ended, the process returns to step S204, and the movement of the observation head 160 and the still image acquisition are repeatedly performed.

In step S210, the third control circuit 310 performs a termination operation. To be more specific, the third control circuit 310 causes the observation apparatus 10 to end the imaging operation performed by the imaging unit 170 and move the observation head 160 to the initial position. In addition, the third control circuit 310 causes the observation apparatus 10 to end the image transmission from the observation apparatus 10 to the display apparatus 40. Thereafter, the process returns to the operation apparatus control.

(Measurement Mode Processing)

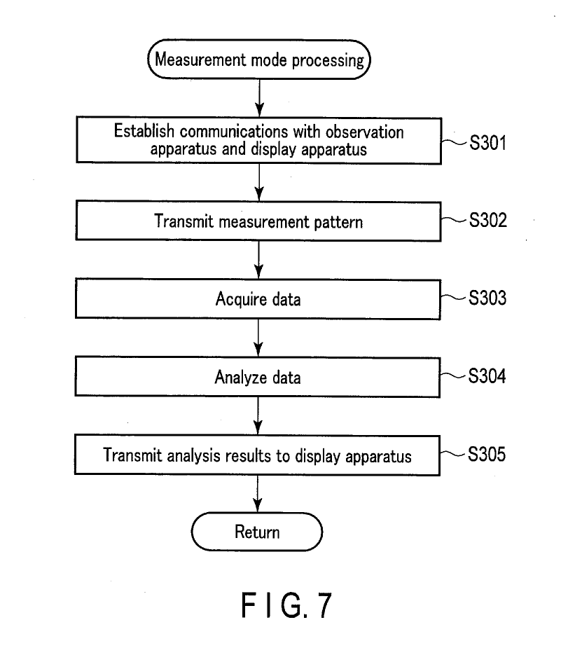

An example of the measurement mode processing performed in step S106 will be described, referring to the flowchart shown in FIG. 7. The measurement mode processing is processing for moving the observation head 160 of the observation apparatus 10 in accordance with a predetermined pattern and causing the imaging unit 170 to take a necessary image.

In step S301, the third control circuit 310 establishes communications among the operation apparatus 30, the observation apparatus 10 and the display apparatus 40.

In step S302, the third control circuit 310 transmits to the observation apparatus 10 a measurement pattern for performing measurement on an image of the sample 50. This measurement pattern includes an imaging position, imaging conditions, etc. The measurement pattern may be prepared by a supplier or a user beforehand and stored in the third recording apparatus 763; alternatively, it may be entered by the user each time measurement is performed. The observation apparatus 10 changes the position of the imaging unit 17 based on the measurement pattern and causes the imaging unit 170 to acquire an image of the sample 50. The observation apparatus 10 performs proper image processing for the acquired image and transmits the processed data to the operation apparatus 30. The observation apparatus 10 may transmit, to the operation apparatus 30, the image data itself or numerical data or the like obtained based on the image data.

In step S303, the third control circuit 310 acquires data based on the image obtained by the imaging, from the observation apparatus 10. In step S304, the third control circuit 310 performs analysis necessary for the measurement, based on the data acquired from the observation apparatus 10. The third control circuit 310 stores the analysis results, for example, in the third recording apparatus 763. The analysis performed then includes, for example, count of the number of cells or cell colonies, determination of the size of a colony, quantitative determination of an emission brightness, etc.

In step S305, the third control circuit 310 transmits analysis results to the display apparatus 40 as the results of the measurement. The analysis results may be transmitted from the operation apparatus 30 directly to the display apparatus 40 using Bluetooth or the like, or may be transmitted to the observation apparatus 10 by using Wi-Fi or the like. After this transmission, the measurement mode processing ends, and the process returns to the operation apparatus control. Upon acquisition of the analysis results, the display apparatus 40 displays the analysis results on the second display unit 470. By this display operation, the user can know the results of the measurement.

During the measurement mode processing, the first display unit 370 of the operation apparatus 30 displays a screen indicating how the processing status is.

(Liquid Operation Mode Processing)

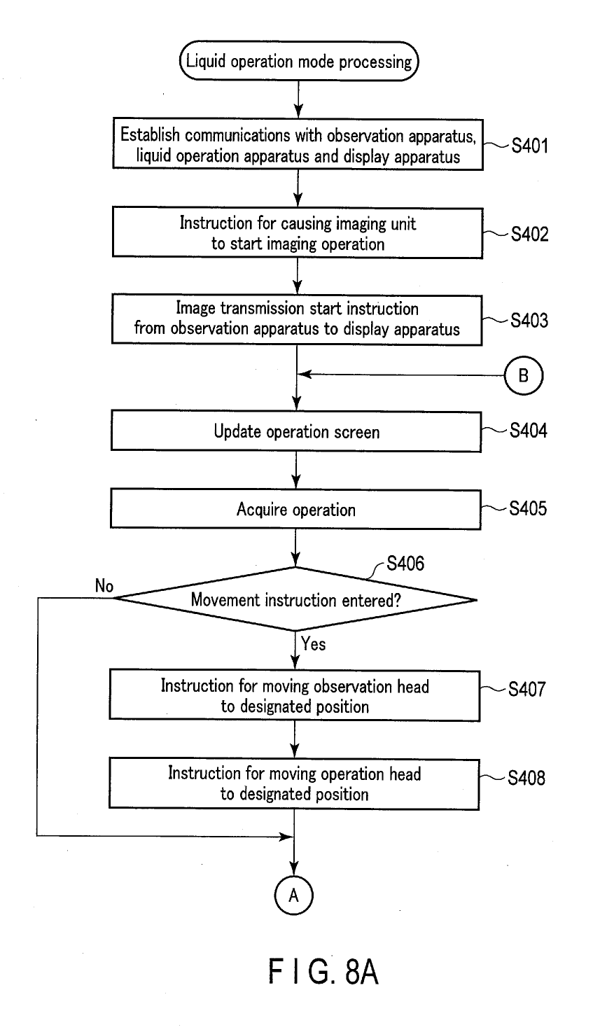

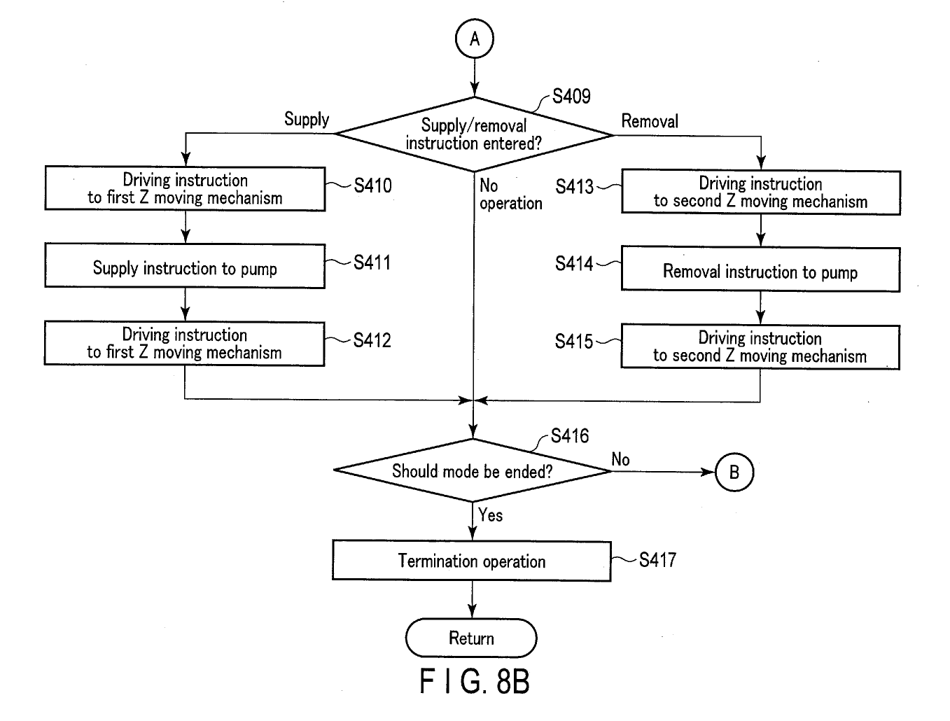

An example of the liquid operation mode processing performed in step S108 will be described, referring to the flowcharts shown in FIG. 8A and FIG. 8B. The liquid operation mode processing is processing in which the observation head 160 of the observation apparatus 10 and the operation head 260 of the liquid operation apparatus 20 are allowed to move in accordance with a user's operation and the liquid operation apparatus 20 is made to supply or remove a liquid in accordance with the user's operation.

In step S401, the third control circuit 310 establishes communications among the operation apparatus 30, the observation apparatus 10, the liquid operation apparatus 20 and the display apparatus 40.

In step S402, the third control circuit 310 supplies the observation apparatus 10 with an instruction for causing the imaging unit 170 to start an imaging operation. As a result, the imaging unit 170 of the observation apparatus 10 starts the imaging operation.

In step S403, the third control circuit 310 supplies the observation apparatus 10 with an instruction for causing the observation apparatus 10 to send the image obtained by the imaging operation to the display apparatus 40. As a result, the observation apparatus 10 starts sending the image obtained by the imaging unit 170 to the display apparatus 40. Upon receipt of the image, the display apparatus 40 displays the received image on the second display unit 470.

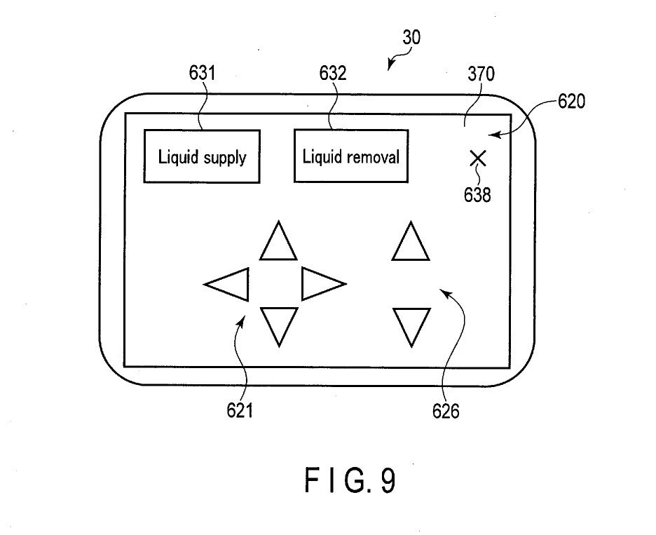

In step S404, the third control circuit 310 updates the operation screen displayed on the first display unit 370. FIG. 9 illustrates an example of the operation screen displayed on the first display unit 370. The operation screen 620 displayed on the first display unit 370 includes a direction indication icon 621 and an up and down indication icon 626. The direction indication icon 621 includes arrow keys for moving the observation head 160 and the operation head 260 in the plus or minus direction on the X-axis, and arrow keys for moving the observation head 160 and the operation head 260 in the plus or minus direction on the Y-axis. The up and down indication icon 626 includes arrow keys for moving the supply unit 272 or removal unit 274 of the operation head 260 upward or downward in the Z-axis direction. The operation screen 620 also includes a liquid supply icon 631 for causing the liquid operation apparatus 20 to supply a liquid to the sample 50 and a liquid removal icon 632 for causing the liquid operation apparatus 20 to remove a liquid from the sample 50. The operation screen 620 further includes an end icon 638 for ending the liquid operation mode.

In step S405, the third control circuit 310 acquires an operation input by the user from the touch panel 380. In step S406, the third control circuit 310 determines whether or not the direction indication icon 621 is touched, namely, whether or not a movement instruction is entered. Unless the movement instruction is entered, the process proceeds to step S409. If the movement instruction is entered, the process proceeds to step S407.

In step S407, the third control circuit 310 supplies the observation apparatus 10 with an instruction for moving the observation head 160 of the observation apparatus 10 in the direction corresponding to the acquired operation. As a result, the observation head 160 moves in the direction indicated by the user. Similarly, in step S408, the third control circuit 310 supplies the liquid operation apparatus 20 with an instruction for moving the operation head 260 of the liquid operation apparatus 20 in the direction corresponding to the acquired operation. As a result, the operation head 260 moves in the direction indicated by the user to bring the operation head 260 and the observation head 160 to be opposed to each other, with the sample 50 interposed therebetween. That is, the observation head 160 and the operation head 260 move together. Subsequently, the process proceeds to step S409.

In addition to the movement of the observation head 160 and operation head 260 in the X-Y plane, the supply unit 272 or removal unit 274 may be moved in the vertical direction in accordance with an input entered with the up and down indication icon 626. Further, the in-focus position of the imaging unit 170 may be moved along the Z-axis in accordance with an input entered with the up and down indication icon 626.

In step S409, the third control circuit 310 determines whether or not the liquid supply icon 631 or liquid removal icon 632 is touched, namely, whether or not a liquid supply or removal instruction is entered. If the liquid supply icon 631 is touched, the process proceeds to step S410. If the liquid removal icon 632 is touched, the process proceeds to step S413. Neither the liquid supply icon 631 nor the liquid removal icon 632 is touched, the process proceeds to step S416.

If the liquid supply icon 631 is touched, in step S410 the third control circuit 310 supplies the liquid operation apparatus 20 with an instruction which causes the supply unit 272 to move closer to the sample 50, i.e., which causes the first Z moving mechanism 285 to lower the supply unit 272. As a result, the first Z moving mechanism 285 moves the supply unit 272 closer to the sample 50.

In step S411, the third control circuit 310 supplies the liquid operation apparatus 20 with an instruction for operating the pump 292 such that a liquid is supplied from the supply unit 272 to the sample 50. As a result, the reagent in the reagent container 294 is supplied to the sample 50.

In step S412, the third control circuit 310 supplies the liquid operation apparatus 20 with an instruction for moving the supply unit 272 away from the sample 50, i.e., for causing the first Z moving mechanism 285 to raise the supply unit 272. As a result, the first Z moving mechanism 285 moves the supply unit 272 away from the sample 50. Subsequently, the process proceeds to step S416.

If the liquid removal icon 632 is touched, the third control circuit 310 supplies the liquid operation apparatus 20 with an instruction (in step S413) which causes the removal unit 274 to move closer to the sample 50, i.e., which causes the second Z moving mechanism 286 to lower the removal unit 274. As a result, the second Z moving mechanism 286 moves the removal unit 274 closer to the sample 50.

In step S414, the third control circuit 310 supplies the liquid operation apparatus 20 with an instruction for operating the pump 292 such that a liquid is removed from the sample 50. As a result, the reagent in the sample 50 is drained to the waste liquid container 295.

In step S415, the third control circuit 310 supplies the liquid operation apparatus 20 with an instruction for moving the removal unit 274 away from the sample 50, i.e., for causing the second Z moving mechanism 286 to raise the removal unit 274. As a result, the second Z moving mechanism 286 moves the removal unit 274 away from the sample 50. Subsequently, the process proceeds to step S416.

In the liquid operation mode processing mentioned above, the Z positions etc. of the removal unit 274 and supply unit 272 are identified based on an image obtained by the imaging unit 170.

In step S416, the third control circuit 310 determines whether or not the liquid operation mode processing should be ended. For example, when an input for ending the liquid operation mode processing is entered by the user, i.e., when the end icon 638 is touched, it is determined that the liquid operation mode processing should be ended. At the time, the process proceeds to step S417. If it is determined that the liquid operation mode processing should not be ended, the process returns to step S404, and the movement of the observation head 160 and operation head 260 and the operation for sharing or removing a reagent are performed.

In step S417, the third control circuit 310 performs a termination operation. To be more specific, the third control circuit 310 causes the observation apparatus 10 to end the imaging operation performed by the imaging unit 170 and to move the observation head 160 to the initial position. In addition, the third control circuit 310 causes the observation apparatus 10 to end the image transmission from the observation apparatus 10 to the display apparatus 40. Further, the third control circuit 310 causes the liquid operation apparatus 20 to move the operation head 260 to the initial position. Thereafter, the process returns to the operation apparatus control.

(Automatic Culture Medium Replacement Mode Processing)

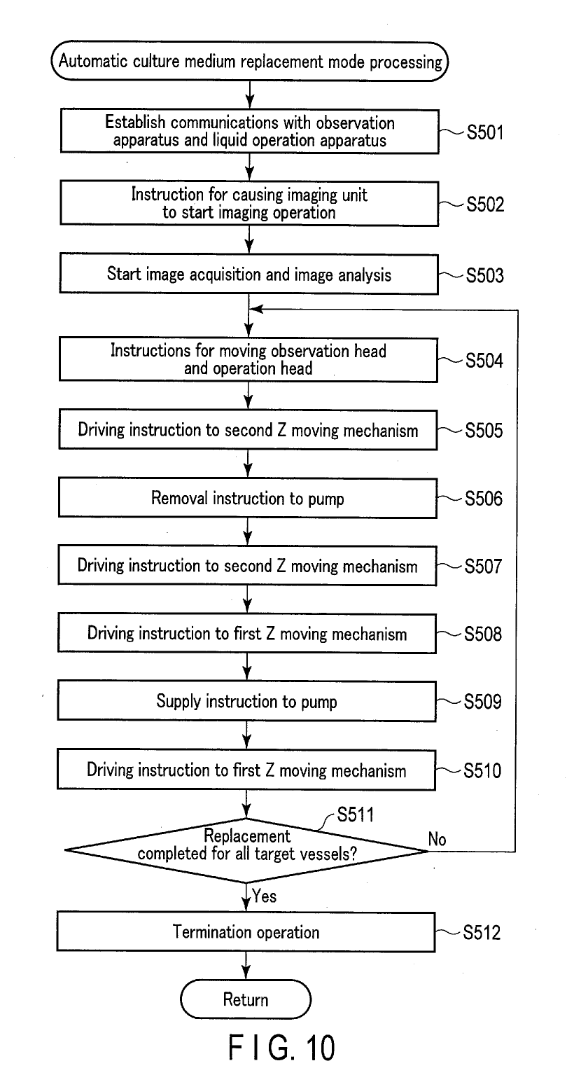

An example of the automatic culture medium replacement mode processing performed in step S110 will be described, referring to the flowchart shown in FIG. 10. The automatic culture medium replacement mode processing is processing in which culture mediums in the sample 50 are replaced without depending on a user's operation.

In step S501, the third control circuit 310 establishes communications among the operation apparatus 30, the observation apparatus 10 and the liquid operation apparatus 20.

In step S502, the third control circuit 310 supplies the observation apparatus 10 with an instruction for causing the imaging unit 170 to start an imaging operation. As a result, the imaging unit 170 of the observation apparatus 10 starts the imaging operation.

In step S503, the third control circuit 310 starts acquiring data pertaining to an image obtained by imaging unit 170. The third control circuit 310 starts analyzing this image data. In the automatic culture medium replacement mode, the third control circuit 310 controls the operations of the observation apparatus 10 and liquid operation apparatus 20, based on the image data.

In step S504, the third control circuit 310 supplies the observation apparatus 10 with an instruction for moving the observation head 160 of the observation apparatus 10 to a proper position of the sample 50, which position is specified based on the analysis results of the image data. Similarly, the third control circuit 310 supplies the liquid operation apparatus 20 with an instruction for moving the operation head 260 of the liquid operation apparatus 20. As a result, the third control circuit 310 moves the observation head 160 and the operation head 260 together to a position proper for the replacement of a culture medium of the sample 50.

The third control circuit 310 may determine a target position to which the observation head 160 and the operation head 260 should be moved, for example by detecting the edges of the vessel 51 and thereby specifying the inside region of the vessel 51. The culture medium replacement can be performed as long as the supply unit 272 and the removal unit 274 are located inside of the vessel 51. Therefore, the third control circuit 310 can determine any position inside the vessel 51 as the target position. Likewise, an inside position near an edge of the vessel 51 or the center of the vessel 51 maybe determined as the target position. The third control circuit 310 may refer to obtained image data and determine that a region where a culture medium is present or a region where cells are present is the inside region of the vessel 51.

In step S505, the third control circuit 310 supplies the liquid operation apparatus 20 with an instruction for operating the second Z moving mechanism 286 to move the removal unit 274 closer to the sample 50. As a result, the second Z moving mechanism 286 moves the removal unit 274 closer to the sample 50.

In step S506, the third control circuit 310 supplies the liquid operation apparatus 20 with an instruction for operating the pump 292 such that the removal unit 274 removes a culture medium from the sample 50. As a result, the culture medium in the sample 50 is drained to the waste liquid container 295.

In step S507, the third control circuit 310 supplies the liquid operation apparatus 20 with an instruction for moving the removal unit 274 away from the sample 50, i.e., for causing the second Z moving mechanism 286 to raise the removal unit 274. As a result, the second Z moving mechanism 286 moves the removal unit 274 away from the sample 50.

In step S508, the third control circuit 310 supplies the liquid operation apparatus 20 with an instruction for operating the first Z moving mechanism 285 to move the supply unit 272 closer to the sample 50. As a result, the first Z moving mechanism 285 moves the supply unit 272 closer to the sample 50.

In step S509, the third control circuit 310 supplies the liquid operation apparatus 20 with an instruction for operating the pump 292 such that a culture medium is supplied from the supply unit 272 to the sample 50. As a result, the culture medium in the reagent container 294 is supplied to the sample 50.

In step S510, the third control circuit 310 supplies the liquid operation apparatus 20 with an instruction for moving the supply unit 272 away from the sample 50, i.e., for causing the first Z moving mechanism 285 to raise the supply unit 272. As a result, the first Z moving mechanism 285 moves the supply unit 272 away from the sample 50. Subsequently, the process proceeds to step S511.

In the automatic culture medium replacement mode processing, the Z positions of the removal unit 274 and the supply unit 272, the amount of culture medium, etc. are determined based on an image obtained by the imaging unit 170. That is, the imaging unit 170 changes the in-focus plane and acquires an image from which the positional relationship between the sample 50 and the removal unit 274 or supply unit 272 can be determined. The third control circuit 310 controls the position of the removal unit 274 or supply unit 272, based on this image. For example, when the supply unit 272 is moved closer to the sample 50, the in-focus position of the imaging unit 170 is adjusted such that a position higher than the cells present in the bottom of the vessel 51 is in focus, not the cells themselves. In this state, the supply unit 272 is lowered until the tip end of the supply unit 272 comes to be confirmed in the image obtained by the imaging unit 170. At this point of time, the distance between the bottom of the vessel 51 and the tip end of the supply unit 272 can be regarded as being several micrometers. The experimental system 1 in the present embodiment, which operates as above, can move the tip end of the supply unit 272 to a position very close to the cells. The operation of changing the in-focus position can be used in the liquid operation mode processing as well. If an observation target such as cells need not be in focus and the tip end of the supply unit 272 or removal unit 274 may be in focus, the Z position of the supply unit 272 or removal unit 274 can be optimally adjusted by changing the in-focus position.

The culture medium at one position can be replaced by performing one operation starting at step S504 to ending at step S510. For example, where the culture medium in a petri dish is replaced, the above operation is performed once. For example, where the culture mediums in a multiwell plate are replaced, the above operation is repeated for the respective wells. In step S511, the third control circuit 310 determines whether or not the culture medium replacement has been completed for all target vessels requiring culture medium replacement, for example, for all the wells of the multiwell plate. If the culture medium replacement has not yet been completed for all target vessels, the process returns to step S504, and the culture medium replacement is repeated. If the culture medium replacement has been completed for all target vessels, the process returns to step S512, and the process proceeds to step S512.

In step S512, the third control circuit 310 performs a termination operation. To be more specific, the third control circuit 310 causes the observation apparatus 10 to end the imaging operation performed by the imaging unit 170 and to move the observation head 160 to the initial position. The third control circuit 310 also causes the liquid operation apparatus 20 to move the operation head 260 to the initial position. Thereafter, the process returns to the operation apparatus control.

### <Operation of Observation Apparatus>

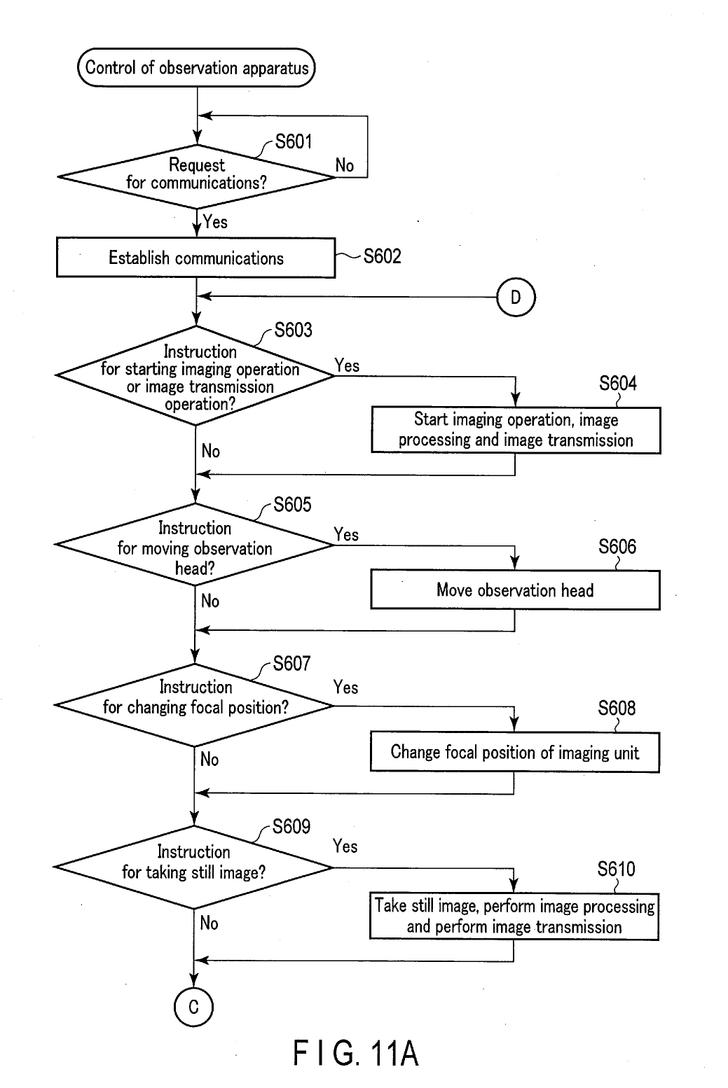

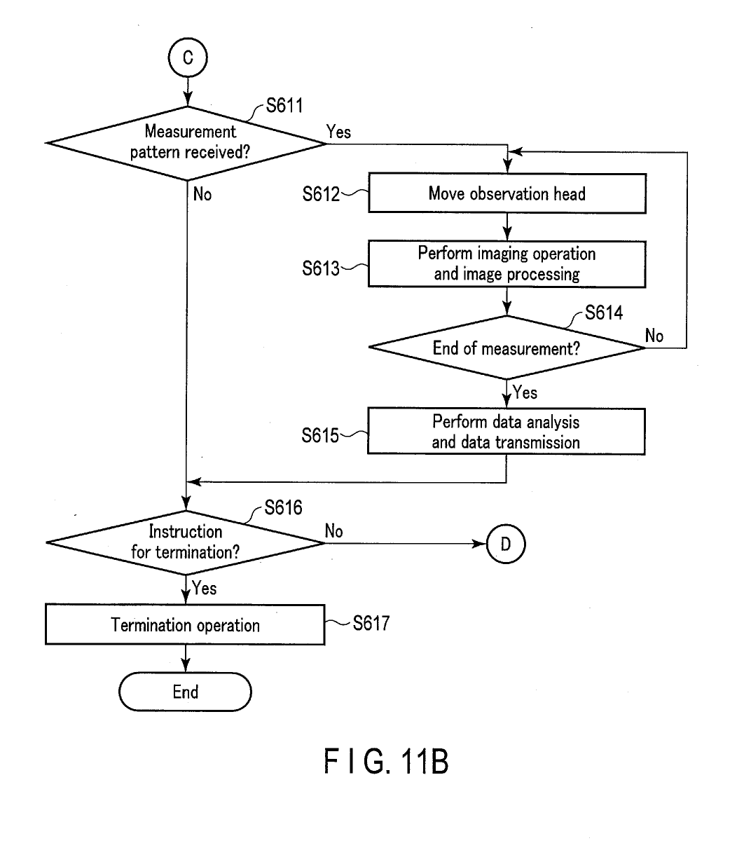

Next, the operation of the observation apparatus 10 will be described, referring to the flowcharts shown in FIGS. 11A and 11B.

In step S601, the first control circuit 110 determines whether or not a request for communications is made by the operation apparatus 30. For example, at the start of each mode processing, the operation apparatus 30 makes a request for communications, as described above. If a request for communications is not made, the process stands by, repeating the processing in step S601. When the request for communications is made, the process proceeds to step S602. For example, the observation apparatus 10 may continue to stand by until the establishment of Wi-Fi communications or the like. For power saving, the observation apparatus 10 may wait for a request for communications, using a low-power-consumption communications means, such as Bluetooth Low Energy, and when the request is made, Wi-Fi communications may be activated.

In step S602, the first control circuit 110 establishes requested communications. For example, in the case of communication using Wi-Fi, the communications with the operation apparatus 30 are established, with the first communication apparatus 152 of the observation apparatus 10 being used as an access point. If required, communications with the display apparatus 40, the liquid operation apparatus 20, etc. may be established.

In step S603, the first control circuit 110 determines whether or not an instruction for starting an imaging operation or an image transmission operation is issued. Unless the instruction for the imaging operation or image transmission operation is issued, the process proceeds to step S605. If the instruction for starting the imaging operation or image transmission operation is issued, the process proceeds to step S604. In step S604, the first control circuit 110 causes the imaging unit 170 to start an imaging operation, performs image processing for the obtained image, and if a request for image transmission is made, transmits the processed image to the requestor. Subsequently, the process proceeds to step S605.

For example, in response to the requests made insteps 5202 and 5203 of the specific position observation mode processing shown in FIG. 6, or in response to the requests made in steps S402 and 5403 of the liquid operation mode processing shown in FIG. 8A, the imaging operation by the imaging unit 170 is started, and the transmission of the resultant image data to the display apparatus 40 is started. In response to the requests made in steps 5502 and 5503 of the automatic culture medium replacement mode processing shown in FIG. 10, the imaging operation by the imaging unit 170 is started, and the data transmission of the resultant image data to the operation apparatus 30 is started.

In step S605, the first control circuit 110 determines whether or not an instruction for moving the observation head 160 is issued. It should be noted that the observation by the experimental system 1 is assumed to include observation of each position of a particular plane in which a sample is distributed. Unless the instruction for moving the observation head 160 is issued, the process proceeds to step S607. If the instruction for moving the observation head 160 is issued, the process proceeds to step S606. In step S606, the first control circuit 110 moves the observation head 160 in response to the received movement instruction. At the time, the position of the observation head 160 can be changed in accordance with the position control performed externally of the observation apparatus 10. The movement instruction can include designation of the coordinates of the position to which the observation head 160 should be moved, designation of the moving method of the observation head 160, and designation of a position determined relative to the sample 50 without reference to the coordinates of the observation head 160. As described above, the operation apparatus 30 may cause the liquid operation apparatus 20 to move the operation head 260 simultaneously when causing the observation apparatus 10 to move the observation head 160. In such a case, the first control circuit 110 extracts a signal relating to the position of the observation head 160 from the control signals including both the signal relating to the position of the observation head 160 and the signal relating to the position of the operation head 260, and controls the operation of the observation moving mechanism 180. Where the target position of the observation head of the observation apparatus 10 and the target position of the operation head 260 of the liquid operation apparatus 20 correspond to each other, the target position of the observation head 160 and the target position of the operation head 260 can be indicated by one control signal. In this case, the first control circuit 110 controls the operation of the observation moving mechanism 180 based on this control signal. Similarly, the second control circuit 210 of the liquid operation apparatus 20 controls the operation of the operation moving mechanism 280 based on the same control signal. Subsequently, the process proceeds to step S607.

For example, in response to the request made in step S206 of the specific position observation mode processing shown in FIG. 6, in response to the request made in step S408 of the liquid operation mode processing shown in FIG. 8A, or in response to the request made in step S504 of the automatic culture medium replacement mode processing shown in FIG. 10, the first control circuit 110 changes the position of the observation head 160.

If the coordinates of the observation head 160 are not designated, as in the case where the movement instruction mentioned above is based on the designation of a relative position to the sample 50, then the observation apparatus 10 (imaging apparatus) has to perform various operations such that the observation head 160 can be moved to the position intended by the movement instruction. For example, where a relative position to the sample 50 is designated, the first control circuit 110 analyzes the position of the sample 50 to specify the indicated position. For example, if the movement instruction is based on the position of the operation head 260 of the liquid operation apparatus 20, then the first control circuit 110 may change the position of the observation head 160 by calculating the target position of the observation head 160 based on the position of the operation head 260. In particular, if the position of the observation head 160 and the position of the operation head 260 are different, the first control circuit 110 can control the position of the observation head 160 (imaging position control) in accordance with the position of the operation head 260 by performing coordinate conversion, numerical inversion or the like with respect to the position of the observation head 160. In such a case, the observation apparatus 10 may specify the position of the operation head 260, based on the light radiating from the second illumination unit 276 of the operation head 260. The observation apparatus 10 may request that the liquid operation apparatus 20 provide information on the position of the operation head 260 or the position of the observation head 160.

The observation head 160 is movable in relation to a specific plane in which a sample is distributed. The operation head 260 of the liquid operation apparatus 20 functions as a substance operation mechanism provided outside the observation apparatus 10 and capable of adding or removing a particular substance. As can be seen from this, the observation apparatus 10 functions as an imaging apparatus for experimental use, which is movable to different positions in a specific plane in which a sample is distributed, and which enables an imaging position to be changed in accordance with the position of a substance operation mechanism configured to add or remove a substance, the imaging apparatus comprising: an imaging unit which generates image data by imaging the sample; an observation moving mechanism which changes the position of the imaging unit; and a control circuit which controls an operation of the observation moving mechanism such that the position of the imaging unit and the substance operation position of the substance operation mechanism change together.

In step S607, the first control circuit 110 determines whether or not an instruction for moving the focal position of the imaging unit 170 is issued. Unless the instruction for moving the focal position is issued, the process proceeds to step S609. If the instruction for moving the focal position is issued, the process proceeds to step S608. In step S608, the first control circuit 110 moves the focal position of the imaging unit 170 in response to the received movement instruction of the focal position. Subsequently, the process proceeds to step S609.

The first control circuit 110 moves the focal position of the imaging unit 170, based on the received movement instruction of the focal position, for example, at the following occasions: in the focusing operation performed at the start of the imaging by the imaging unit 170; in the operation of moving the supply unit 272 or removal unit 274 in the Z-axis direction in steps 410 to 415 of the liquid operation mode processing shown in FIG. 8B; or in the operation of moving the supply unit 272 or removal unit 274 in the Z-axis direction in steps 505 to 510 of the culture medium replacement mode processing shown in FIG. 10.

In step S609, the first control circuit 110 determines whether or not an instruction for taking a still image is issued. Unless the instruction for taking the still image is issued, the process proceeds to step S611. If the instruction for taking a still image is issued, the process proceeds to step S610. In step S610, the first control circuit 110 causes the imaging unit 170 to take a still image, performs image processing for the obtained image, and if a request for image transmission is made, transmits the processed image to the requestor. Subsequently, the process proceeds to step S611.

For example, in response to a request made in step S208 of the specific position observation mode processing shown in FIG. 6, the first control circuit 110 causes the imaging unit 170 to take a still image, performs image processing, and transmits the image subjected to the image processing to the display apparatus 40.

In step S611, the first control circuit 110 determines whether or not a measurement pattern for a measurement operation is received. Unless the measurement pattern is received, the process proceeds to step S616. If the measurement pattern is received, for example, in response to the operation in step S302 of the measurement mode processing shown in FIG. 7, the process proceeds to step S612.

In step S612, the first control circuit 110 moves the observation head 160 in response to the received information on the measurement pattern. In step S613, the first control circuit 110 causes the imaging unit 170 to perform an imaging operation, based on the received information on the measurement pattern, and performs image processing for the obtained image.

In step S614, the first control circuit 110 determines whether or not all measurements based on the measurement pattern come to an end. Unless all measurements end, the process returns to step S612, and imaging and image processing are repeated while changing the position of the imaging unit 170. If the measurements end, the process proceeds to step S615.

In step S615, the first control circuit 110 performs the necessary data analysis based on the obtained images, and transmits data on the analysis results, images etc. to the operation apparatus 30. Subsequently, the process proceeds to step S616.

In step S616, the first control circuit 110 determines whether or not an instruction for termination is issued. Unless the instruction for termination is issued, the process returns to step S603, and the processing mentioned above is repeated. If the instruction for termination is issued based on, for example, the termination operation in step S210 of the specific position observation mode processing shown in FIG. 6, the termination operation in step S417 of the liquid operation mode processing shown in FIG. 8B, or the termination operation in step S512 of the automatic culture medium replacement mode processing shown in FIG. 10, the process proceeds to step S617.

In step S617, the first control circuit 110 performs a termination operation in which the imaging operation by the imaging unit 170 is ended and the observation head 160 is moved back to the initial position. Then, the observation apparatus control is terminated.

As described above, the first control circuit 110 operates each portion of the observation apparatus 10 based on, for example, various instructions acquired from the operation apparatus 30.

<Operation of Liquid Operation Apparatus>

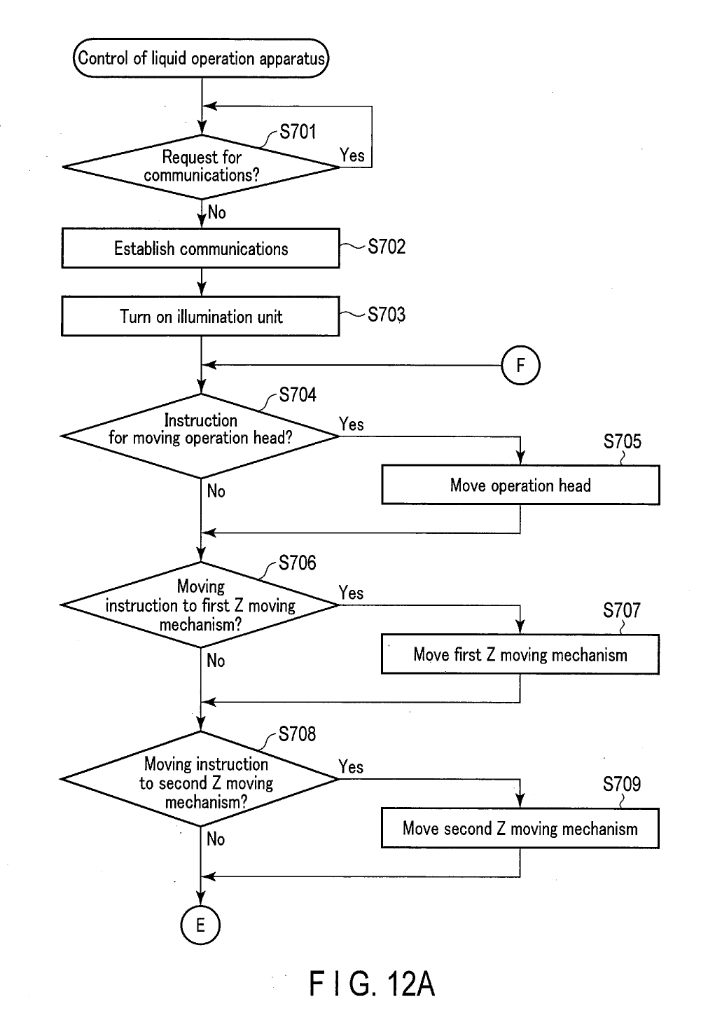

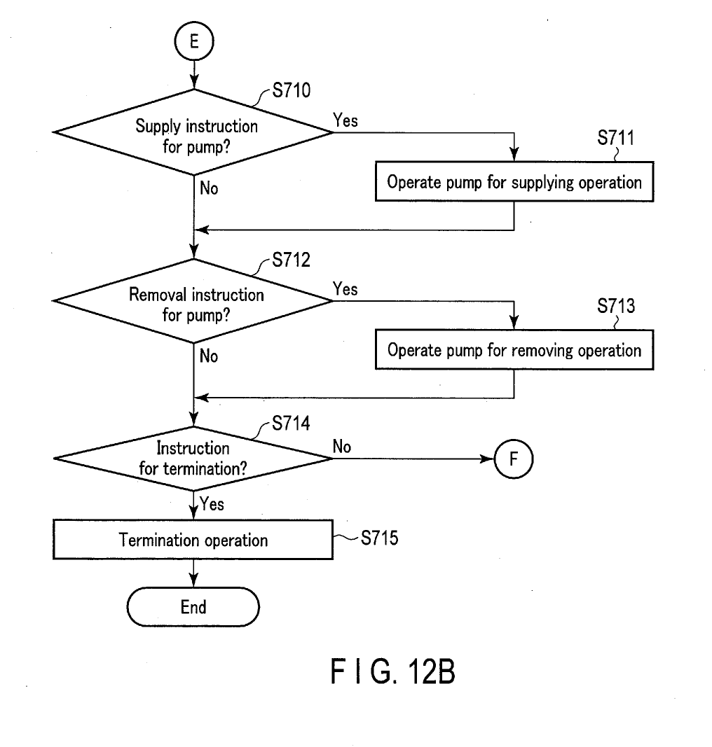

Next, the operation of the liquid operation apparatus 20 will be described, referring to the flowcharts shown in FIGS. 12A and 12B.

In step S701, the second control circuit 210 determines whether or not a request for communications is made. For example, at the start of each mode processing, the operation apparatus 30 makes a request for communications, as described above. If a request for communications is not made, the process stands by, repeating the processing in step S701. If the request for communications is made, the process proceeds to step S702.

In step S702, the second control circuit 210 establishes requested communications. For example, in the case of communication using Wi-Fi, the communications with the observation apparatus 10 are established, with the first communication apparatus 152 of the observation apparatus 10 being used as an access point. Alternatively, the communications with the operation apparatus 30 etc. may be established using Bluetooth.

In step S703, the second control circuit 210 turns on the second illumination unit 276.

In step S704, the second control circuit 210 determines whether or not an instruction for moving the operation head 260 is issued. Unless the instruction is issued, the process proceeds to step S706. If the instruction for moving the operation head 260 is issued, the process proceeds to step S705. In step S705, the second control circuit 210 moves the operation head 260 in response to the movement instruction. Subsequently, the process proceeds to step S706.

For example, in response to the instruction issued in step S408 of the liquid operation mode processing shown in FIG. 8A, or in response to the instruction issued in step S504 of the automatic culture medium replacement mode processing shown in FIG. 10, the second control circuit 210 changes the position of the operation head 260.

In step S706, the second control circuit 210 determines whether or not an instruction for moving the first Z moving mechanism 285 is issued. Unless the instruction for moving the first Z moving mechanism is issued, the process proceeds to step S708. If the instruction for moving the first Z moving mechanism 285 is issued, the process proceeds to step S707. In step S707, the second control circuit 210 operates the first Z moving mechanism 285 based on the movement instruction. Subsequently, the process proceeds to step S708.

For example, in response to the operation instruction issued in step S410 or step S412 of the liquid operation mode processing shown in FIG. 8A, or in response to the operation instruction issued in step S508 or step S510 of the automatic culture medium replacement mode processing shown in FIG. 10, the second control circuit 210 changes the position of the first Z moving mechanism 285.

In step S708, the second control circuit 210 determines whether or not an instruction for moving the second Z moving mechanism 286 is issued. Unless the instruction for moving the second Z moving mechanism is issued, the process proceeds to step S710. If the instruction for moving the second Z moving mechanism 286 is issued, the process proceeds to step S709. In step S709, the second control circuit 210 moves the second Z moving mechanism 286 based on the movement instruction. Subsequently, the process proceeds to step S710.

For example, in response to the operation instruction issued in step S413 or step S415 of the liquid operation mode processing shown in FIG. 8B, or in response to the operation instruction issued in step S505 or step S507 of the automatic culture medium replacement mode processing shown in FIG. 10, the second control circuit 210 operates the second Z moving mechanism 286.

In step S710, the second control circuit 210 determines whether or not a supply instruction of the pump is issued. Unless the supply instruction is issued, the process proceeds to step S712. If the supply instruction is issued, the process proceeds to step S711. In step S711, the second control circuit 210 operates the pump 292 to supply a reagent. Subsequently, the process proceeds to step S712.

For example, in response to the supply instruction issued in step S411 of the liquid operation mode processing shown in FIG. 8B, or in response to the supply instruction issued in step S509 of the automatic culture medium replacement mode processing shown in FIG. 10, the second control circuit 210 operates the pump 292.

In step S712, the second control circuit 210 determines whether or not a removal instruction to the pump is issued. Unless the removal instruction is issued, the process proceeds to step S714. If the removal instruction is issued, the process proceeds to step S713. In step S713, the second control circuit 210 operates the pump 292 to remove a reagent. Subsequently, the process proceeds to step S714.

For example, in response to the removal instruction issued in step S414 of the liquid operation mode processing shown in FIG. 8B, or in response to the instruction issued in step S506 of the automatic culture medium replacement mode processing shown in FIG. 10, the second control circuit 210 operates the pump 292.

In step S714, the second control circuit 210 determines whether or not an instruction for termination is issued. Unless the instruction for termination is issued, the process returns to step S704, and the processing mentioned above is repeated. If the instruction for termination is issued based on, for example, the termination operation in step S417 of the liquid operation mode processing shown in FIG. 8B, or the termination operation in step S512 of the automatic culture medium replacement mode processing shown in FIG. 10, the process proceeds to step S715.

In step S715, the second control circuit 210 performs a termination operation in which the operation of the pump 292 is ended and the operation head 260 is moved back to the initial position. Then, the liquid operation apparatus control is terminated.

As described above, the second control circuit 210 operates each portion of the liquid operation apparatus 20 based on, for example, various instructions acquired from the operation apparatus 30.

<Operation of Display Apparatus>

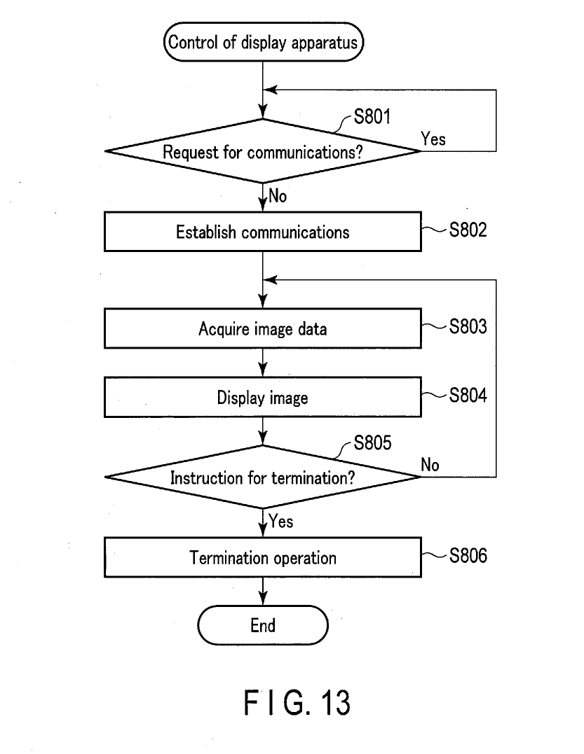

How the display apparatus 40 operates will be described with reference to the flowchart shown in FIG. 13.

In step S801, the fourth control circuit 410 determines whether or not a request for communications is made. For example, at the start of each mode processing, the operation apparatus 30 makes a request for communications, as described above. Unless the request for communications is made, the process stands by, repeating the processing in step S801. If the request for communications is made, the process proceeds to step S802.

In step S802, the fourth control circuit 410 establishes requested communications. For example, in the case of communication using Wi-Fi, the communications with the observation apparatus 10 are established, with the first communication apparatus 152 of the observation apparatus 10 being used as an access point. Alternatively, the communications with the operation apparatus 30 etc. may be established using Bluetooth.

In step S803, the fourth control circuit 410 acquires image data from the observation apparatus 10. In step S804, the fourth control circuit 410 causes the second display unit 470 to display an image, based on the acquired image data. The fourth control circuit 410 may acquire image data from the operation apparatus 30 and cause the second display unit 470 to display an image based on the image data.

In step S805, the fourth control circuit 410 determines whether or not an instruction for termination is issued. Unless the instruction for termination is issued, the process returns to step S803, and the image display processing mentioned above is repeated. If the instruction for termination is issued based on the termination operation in step S210 of the specific position observation mode processing shown in FIG. 6, the termination operation in step S417 of the liquid operation mode processing shown in FIG. 8B, or the termination operation in step S512 of the automatic culture medium replacement mode processing shown in FIG. 10, the process proceeds to step S806.

In step S806, the fourth control circuit 410 performs a termination operation in which, for example, the second display unit 470 is made to stop displaying an image. Then, the display apparatus control is terminated.

As described above, the fourth control circuit 410 causes the second display unit 470 to display an image, based on image data acquired from the observation apparatus 10 or the operation apparatus 30.

Where the user installs, for example, the observation apparatus 10 and liquid operation apparatus 20 in the incubator and statically places the sample 50 on the transparent plate 102 of the observation apparatus 10, the experimental system 1 described above enables, for example, the following. The user can observe the sample 50 statically placed in the incubator. The user can add a desired amount of desired reagent to a desired position of the sample 50 statically placed in the incubator at desired timing. The user can perform culture medium replacement (required during culture of cells) for the sample 50 statically placed in the incubator. Because of this, the user's operations can be reduced, including taking the sample in and out from the incubator and the manual replacement of a culture medium. As a result, the efficiency of the experiment using cells, the degree of freedom of the experiment method, etc. can be improved.

[Modifications]

The measurement mode was described, referring to the example in which a measurement pattern is transmitted from the operation apparatus 30 to the observation apparatus 10 and the observation apparatus 10 operates based on this measurement pattern. However, this is not restrictive, and the operation apparatus 30 may instruct operations of the observation apparatus 10 one by one. The automatic culture medium replacement mode was described, referring to the example in which the operation apparatus 30 instructs operations of the observation apparatus 10 and liquid operation apparatus 20 one by one, and the observation apparatus 10 and the liquid operation apparatus 20 operate based on the instructions. However, this is not restrictive. An operation pattern may be transmitted from the operation apparatus 30 to both the observation apparatus 10 and the liquid operation apparatus 20, and the observation apparatus 10 and the liquid operation apparatus 20 may operate based on this operation pattern. The measurement pattern or operation pattern mentioned above may be stored in the observation apparatus 10 or liquid operation apparatus 20. In this case, the operation apparatus 30 merely designates a measurement pattern or an operation pattern to be used.

In either operation, the positions of the observation head 160 and operation head 260 may be controlled based on an image acquired by the imaging unit 170. In this case, the position of the operation head 260 may be specified based on the illumination light of the second illumination unit 276 of the operation head 260, and the positions of the observation head 160 and operation head 260 may be controlled such that these heads are opposed to each other. For example, when the position of the observation head 160 is changed, the position of the operation head 260 may be changed such that the operation head 260 is opposed to the observation head 160. Alternatively, when the position of the operation head 260 is changed, the position of the observation head 160 may be changed such that the observation head 160 is opposed to the operation head 260.

Feedback control may be performed for the positional adjustment between the observation head 160 and the operation head 260. For example, the position of the operation head 260 may be acquired by the observation apparatus 10, and the observation head 160 may be moved to the position corresponding to that of the operation head 260. Alternatively, the position of the operation head 260 may be acquired by the observation apparatus 10, and the observation apparatus 10 may issue an instruction to the liquid operation apparatus 20 such that the operation head 260 should be moved to the position corresponding to that of the observation head 160. Conversely, the liquid operation apparatus 20 may acquire the position of the observation head 160.

Open-loop control may be performed for the positional adjustment between the observation head 160 and the operation head 260. In the open-loop control, a specific position is designated, and the observation head 160 and the operation head 260 are controlled and moved to the same position. In this case, the transparent plate 102 and the vessel 51 are arranged, for example, such that their corners are fitted with each other. As a result, the operation apparatus 30 can hold information on the coordinates of the vessel 51. The coordinates of the vessel 51 are recorded in the third recording apparatus 763 or the like, and the third control circuit 310 transmits a signal for moving the observation head 160 to a designated position in the vessel 51. Similarly, the third control circuit 310 transmits an instruction for moving the operation head 260 to the designated position in the vessel 51. Where the vessel 51 is arranged at a specific position, the position of the observation head 160 and the moving direction of the operation head 260 can be limited to one of the X direction and the Y direction.

The position of the observation head 160 and the position of the operation head 260 can be controlled by translating the positions into actuator driving amounts of either the observation moving mechanism 180 or the operation moving mechanism 280. This translation may be predetermined such that the operation apparatus 30 can perform this translation. This translation may be performed by either the observation apparatus 10 or the liquid operation apparatus 20, based on the difference between the current position of a head and the target position of the head.

The mode for adding a predetermined amount of predetermined reagent to a predetermined position in accordance with a predetermined pattern may be prepared not only for the culture medium replacement mode but also for the addition of a reagent to the sample 50.

The above-mentioned embodiment was described, referring to the case where the second illumination unit 276 is used as an illumination light source only when the supply unit 272 or removal unit 274 operates, such as when a reagent is added or when a culture medium is replaced. This is not restrictive, and the second illumination unit 276 may be used as an illumination light source in the specific position observation mode and in the measurement mode.

The above-mentioned embodiment was described, referring to the case where an image acquired by the observation apparatus 10 is displayed on the display apparatus 40, but this is not restrictive. The image acquired by the observation apparatus 10 maybe displayed on the first display unit 370 of the operation apparatus 30.

With respect to the automatic culture medium replacement, the experimental system 1 may be configured, for example, such that a specific culture medium 53 is replaced at specific times under program control. If the observation by the observation head 160 is performed simultaneously with the culture medium replacement, how the state of cells is at the time of the automatic culture medium replacement can be recorded. As a result, a database can be prepared showing how the state of cells is at the time of the automatic culture medium replacement.

When the position of the observation head 160 or the position of the operation head 260 is changed in response to a user's operation, the position of the observation head 160 or operation head 260 can be specified based on the amount in which the actuator of the observation moving mechanism 180 or operation moving mechanism 280 is driven. To be more specific, the driving amount of the actuator is converted into coordinates representing the position of the observation head 160 or operation head 260. Using the coordinates obtained by this conversion, the observation apparatus 10, the liquid operation apparatus 20 or the operation apparatus 30 can control the operation of the observation head 160 or operation head 260. The interrelating operations of the observation head 160 and operation head 260 can be controlled using the common coordinates.

An operation by the user can be performed using such an interface as the touch panel provided for the operation apparatus 30. In this case, if the relationship between the position designated by the user and the coordinates of the observation head 160 or operation head 260 is known beforehand, the position of the observation head 160 or operation head 260 can be easily controlled. Therefore, the observation head 160 and the operation head 260 can be controlled such that the observation head 160 and the operation head 260 move together. The vessel 51 may be a petri dish, a flask or any container that is not a multiwall plate. If the coordinate origin of the vessel 51 is determined and the positional relationships between this origin and the coordinates of the observation head 160 and the operation head 260 are known, the positions of the observation head 160 and operation head 260 can be easily controlled. In other words, the interrelating control of the observation head 160 and the operation head 260 can be performed using the origin.

Of the techniques described in connection with the above embodiments, the controls described with reference to flowcharts are realized as programs. The programs can be stored in a recording medium or a recording apparatus. The programs can be recorded in the recording medium or recording apparatus in various ways. They may be recorded at the time of shipping a product, they can be recorded using a distributed recording medium, or they can be downloaded from the Internet.

In the experimental system 1, it is important for the observation apparatus 10 and the liquid operation apparatus 20 to operate in relation with each other for a specific position. If the observation apparatus 10 and the liquid operation apparatus 20 fail to operate properly, the user may have to take appropriate measures to make up for the failure. In such a case, the user may change the target positions of the observation head 160 and operation head 260 to other positions on purpose. These operations can be recorded, and relationships between failures and operations for making up for the failures and information on various positions and situations regarding the differences between operations satisfying the user and operations dissatisfying the user can be accumulated. By using these kinds of information as learning data, artificial intelligence can be made to learn operations of the experimental system 1. By making use of the artificial intelligence that learns the operations, more appropriate operations, elimination of errors, more precise control etc. can be realized.

The above embodiments of the present invention encompass the following inventions:
[1] An imaging apparatus for experimental use, which is movable to different positions in a specific plane in which a sample is distributed, and which enables an imaging position to be changed in accordance with a position of an external substance operation mechanism configured to add or remove a substance, said imaging apparatus comprising:

an imaging unit which generates image data by imaging the sample;

an observation moving mechanism which changes a position of the imaging unit; and

a control circuit which controls an operation of the observation moving mechanism such that the position of the imaging unit and a substance operation position of the substance operation mechanism change together.
[2] The imaging apparatus according to [1] wherein light emitted from the substance operation position, and the controller controls an operation of the observation moving mechanism, based on a light signal emitted from the substance operation position.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An imaging apparatus for experimental use, which is movable to different positions in a specific plane in which a sample is distributed, and which enables an imaging position to be changed in accordance with a position of an external substance operation mechanism configured to add or remove a substance, the imaging apparatus comprising:
an imaging unit which generates image data by imaging the sample;
an observation moving mechanism which changes a position of the imaging unit; and
a control circuit which controls an operation of the observation moving mechanism such that the position of the imaging unit and a substance operation position of the substance operation mechanism change together.

2. The imaging apparatus according to claim 1, wherein the control circuit extracts a signal relating to the position of the imaging unit from control signals including the signal relating to the position of the imaging unit and a signal relating to the substance operation position, and controls the operation of the observation moving mechanism.

3. The imaging apparatus according to claim 1, wherein the control circuit controls the operation of the observation moving mechanism, based on a single control signal relating to both the position of the imaging unit and the substance operation position.

4. The imaging apparatus according to claim 1, wherein the control circuit determines the position of the imaging unit, based on the substance operation position, and controls the operation of the observation moving mechanism.

5. The imaging apparatus according to claim 4, wherein the control circuit determines the position of the imaging unit, based on light emitted from the substance operation mechanism.

6. The imaging apparatus according to claim 4, wherein the control circuit requests that an apparatus including the substance operation mechanism provide information relating to the position of the imaging unit, when a control signal relating to the substance operation position is received by the control circuit.

7. An experimental system comprising:
an imaging unit which generates image data by imaging a sample;
an observation moving mechanism which changes a position of the imaging unit;
a supply and removal unit which supplies a liquid to the sample or removes the liquid from the sample;
an operation moving mechanism which changes a position of the supply and removal unit;
a pump which moves the liquid through the supply and removal unit; and
a control circuit which controls an operation of the observation moving mechanism and an operation of the operation moving mechanism such that the position of the imaging unit and the position of the supply and removal unit change together, and which controls an operation of the pump in accordance with the position of the imaging unit and the position of the supply and removal unit.

8. The experimental system according to claim 7, wherein the control circuit controls the operation of the observation moving mechanism and the operation of the operation moving mechanism, using the image data.

9. The experimental system according to claim 7, wherein
the liquid is a culture medium used for culturing cells contained in the sample, and
the control circuit controls the operation of the observation moving mechanism, the operation of the operation moving mechanism and the operation of the pump, such that a culture medium is removed from the sample by means of the supply and removal unit and then a culture medium is supplied to the sample, thereby performing culture medium replacement.

10. The experimental system according to claim 7, wherein the imaging unit and the supply and removal unit are arranged to be opposed to each other, with the sample interposed therebetween.

11. The experimental system according to claim 10, further comprising:
an illumination unit which is moved by the operation moving mechanism, together with the supply and removal unit, and which illuminates the sample,
wherein the control circuit controls the operation of the observation moving mechanism and the operation of the operation moving mechanism, based on an image taken by the imaging unit and relating to illumination light radiating from the illumination unit.

12. A method for controlling an experimental system which includes: an imaging unit which generates image data by imaging a sample; an observation moving mechanism which changes a position of the imaging unit; a supply and removal unit which supplies a liquid to the sample or removes a liquid from the sample; an operation moving mechanism which changes a position of the supply and removal unit; and a pump which moves the liquid through the supply and removal unit, the method comprising:
controlling an operation of the observation moving mechanism and an operation of the operation moving mechanism such that the position of the imaging unit and the position of the supply and removal unit change together; and
controlling an operation of the pump in accordance with the position of the imaging unit and the position of the supply and removal unit.
